(19) United States
(12) Patent Application Publication
(10) Pub. No.: US 2020/0066394 A1
(43) Pub. Date: Feb. 27, 2020

(54) MEDICAL IMAGE MANAGEMENT SYSTEM, MEDICAL IMAGE ACQUISITION APPARATUS, MEDICAL IMAGE DATA FILE ACQUISITION METHOD AND MEDICAL IMAGE MANAGEMENT SERVER

(71) Applicant: Olympus Corporation, Tokyo (JP)

(72) Inventors: Tetsuya TOYODA, Tokyo (JP); Masaru IKEDA, Fujimi-shi (JP); Hideaki YOSHIDA, Tokyo (JP); Kazuhiko OSA, Tokyo (JP); Osamu NONAKA, Kanagawa (JP)

(21) Appl. No.: 16/550,117

(22) Filed: Aug. 23, 2019

(30) Foreign Application Priority Data
Aug. 27, 2018 (JP) 2018-158410

(51) Int. Cl. G16H 30/20; G16H 10/60; G16H 15/00

(52) U.S. Cl. CPC G16H 30/20; G06T 7/0012; G16H 15/00; G16H 10/60

(57) ABSTRACT

A medical image management system creating, sharing and managing a medical image data file includes: an information terminal apparatus configured to transmit medical-related particular-use information about a particular individual; an image pickup apparatus configured to acquire image data; and a processor including hardware configured to associate medical service-related data obtained by receiving the medical-related particular-use information with the image data to generate a medical image data file.

This application claims the benefit of Japanese Application No. 2018-158410 filed in Japan on Aug. 27, 2018, the contents of which are incorporated herein by this reference.

### BACKGROUND OF THE INVENTION

### 1. Field of the Invention

The present invention relates to an image management system facilitating management of image data when photographing is performed in response to a request to photograph a particular target object, and the image management system includes an apparatus configured to make a photographing request, an apparatus configured to receive the photographing request, an apparatus configured to manage image data and the like as a result of photographing being performed, and the like. The present invention also includes a method for cooperation among the respective apparatuses in the image management system. Of course, an invention related to a program medium configured to control the apparatuses is also included.

As an embodiment, a medical system is described as a representative of photographing systems for such use. Especially, a medical image management system configured to create, share and manage a medical image data file, a medical image acquisition apparatus configured to acquire a medical image data file that can be handled in the medical image management system, a method for acquiring the medical image data file, and a medical image management server are described as representative examples.

### 2. Description of Related Art

Recently, information terminal apparatuses capable of easily handling various kinds of electronic data including electronic image data, which are provided with a communication function and are in a small-sized and portable form, have come into common use.

The information terminal apparatuses of this kind are configured to be capable of directly performing mutual communication with other information terminal apparatuses of the same kind to perform exchange of various kinds of electronic data and the like and capable of accessing a file server or the like at a remote location via an existing network to perform communication (transmission/reception) of various kinds of electronic data with the file server or the like.

In this case, in order to mutually connect the plurality of information terminal apparatuses and the file server in which various kinds of electronic data and the like are accumulated, via the existing network, various kinds of ICTs (information and communication technologies) are used. Consequently, a network system is configured in which it is possible to handle various electronic data and the like and share the electronic data and the like among the respective information terminal apparatuses. Network systems of this kind have been put to practical use and have rapidly spread in various kinds of fields.

As one form of the network systems of this kind, for example, a data management system has been put to practice in a medical field, the data management system being constructed so that medical workers and the like (for example, doctors, nurses, various kinds of professional technicians (equipment handling qualified workers and the like) and various kinds of medical-related qualified workers such as certified care workers) can access a file server installed in a medical facility such as a hospital via a network using information terminal apparatuses to perform mutual communication with the file server and share medical-related information (for example, electronic medical record information and patient information).

More specifically, for example, a hospital information system (HIS) is a system for centrally managing various kinds of systems for performing hospital work, such as a medical care acceptance/appointment system, an electronic medical record system, a medical accounting system and a management system for management of entering/leaving the hospital and a pharmacy.

A radiology information system (RIS) is a system for managing work related to a radiology department in a hospital such as radiological examination order management, examination appointment management, management of patient information and photographing implementation information for a photographing apparatus, and film management.

In the recent medical field, medical image acquisition apparatuses in various kinds forms in which medical images are treated as electronic digital image data have been put to practical use and have spread.

Here, more specifically, the medical image includes, for example, a radiograph (X-ray image), a computed tomography (CT) image, an MRI (magnetic resonance imaging) image, an ultrasonic tomographic image, an angiographic image, an endoscopic image, a thermographic image and a microscope image. In addition, general photographing images and the like acquired using a common communication-function-equipped image pickup apparatus (such as a digital camera) or an image acquisition apparatus such as an image-pickup/communication-function-equipped information terminal apparatus (a smartphone, a tablet PC or the like) may be included.

Medical image data files in various forms acquired by the various kinds of medical image acquisition apparatuses are centrally managed by a medical image management system or the like, for example, a picture archiving and communication system (PACS).

This kind of medical image management system has a function of receiving a plurality of medical image data files acquired by a plurality of medical image acquisition apparatuses, information terminal apparatuses and the like via a network or the like, storing the medical image data files into an image file server included in the system to create a database, a function of, in response to requests from various kinds of terminal apparatuses, transferring corresponding data files, among the various kinds of digital data files recorded in the image file server, to the request-source terminal apparatuses, and the like. Note that the terminal apparatuses receiving the data files display the received data files in a predetermined form.

As one of typical network communication standards for transmitting/receiving digital image data among a plurality of medical image acquisition apparatuses, a plurality of information terminal apparatuses and the like in this kind of medical image management system, for example, a standard called the DICOM (digital imaging and communications in medicine) standard has been recently widespread. Medical image observation apparatuses for viewing (referring to) and managing objects (image data) in a medical image data format compliant with the DICOM standard (hereinafter referred to as a DICOM-compliant data format) have been widespread in the medical field.

Among electronic data handled in the medical field, however, various kinds of electronic data exist in addition to medical image data, and, among the electronic data, there are a lot of objects (electronic data) in a data format that is not compliant with the DICOM standard (a DICOM-noncompliant data format).

Therefore, for example, a medical image management system for enabling various kinds of objects (electronic data files) in DICOM-noncompliant data formats to be viewed (referred to) using a medical image observation apparatus for viewing (referring to) and managing objects (electronic image data) in a DICOM-compliant data format, and managing the objects is proposed in various forms, for example, by Japanese Patent Application Laid-Open Publication No. 2016-35717.

The medical image management system disclosed by Japanese Patent Application Laid-Open Publication No. 2016-35717 and the like makes it possible to perform management of various kinds of electronic data according to a management system compliant with the DICOM standard by converting medical objects in DICOM-noncompliant data formats to medical objects in a DICOM-compliant data format.

It is made possible thereby to treat data that is noncompliant with a particular standard, such as medical care information providing documents, medical certificates, pathological examination results, hospital papers, referral letters from other hospitals, copies of insurance cards and electronic medical records, under the particular standard. However, because of being noncompliant, there is a problem that, when a requirement of the particular standard is not satisfied, it is not possible to make a recovery.

As conventional information terminal apparatuses, image acquisition apparatuses in various kinds of forms, for example, image pickup apparatuses, such as a digital camera equipped with an image pickup function of acquiring electronic image data and a communication function, and image-pickup/communication-function-equipped information terminal apparatuses such as a smartphone and a tablet PC, and the like are widespread.

The image-pickup/communication-function-equipped information terminal apparatuses (image acquisition apparatuses) are configured to be capable of acquiring and recording an image data file compliant with a predetermined file format by performing an image pickup operation.

As a representative file format of an image data file handled by the information terminal apparatuses (image acquisition apparatuses) of this kind, for example, a format called the Exif (exchangeable image file format) standard is widespread. In the Exif standard, it is specified that one image data file is constructed by an image data body and metadata, which is accompanying information accompanying the image data body. The image standard is normally not conscious of the particular standard, and there may be a case where the image standard does not satisfy, for example, standards for medical images.

Usually, a standard for images of common terminal apparatuses (general image acquisition apparatuses) may not satisfy, for example, standards for medical images.

In general, it is assumed that a medical image officially recognized as patient information is only image information that is acquired based on specified settings and format by a predetermined medical-related qualified worker (a medical worker) using a predetermined medical image apparatus in response to information including a plurality of instructions from a doctor.

Therefore, an image of an affected part, a symptom or the like acquired by a person without a predetermined medical-related qualification (for example, a caregiver such as a patient's family member or close relative or the patient himself/herself; hereinafter, such a person will be referred to as a non-medical worker) using, for example, an image pickup apparatus in a general form (for example, a digital camera) or a communication terminal apparatus that is generally widespread (for example, a smartphone or a tablet PC) normally cannot be treated as an official medical image.

Recently, however, small-size communication terminal apparatuses provided with an image pickup function capable of easily acquiring a high-resolution image as well as a communication function, which is suitable to carry (more specifically, for example, a smartphone and a tablet PC) have been generally widespread.

Furthermore, among image pickup apparatuses such as digital cameras, such that are provided with a communication function have been in practical use. In description below, such communication-function-equipped pickup apparatuses, image-pickup-function-equipped communication apparatuses and the like will be generically referred to as information terminal apparatuses, image acquisition apparatuses, general image acquisition apparatuses or the like.

As described above, in recent years, an environment in which anyone can acquire a high-resolution image anytime, and acquired image data can be easily shared with others having similar information communication terminal apparatuses has been established.

Therefore, for example, if not only image data acquired by medical workers but also image data acquired by non-medical workers, for example, using their own information terminal apparatuses in a general form (for example, digital cameras, smartphones and tablet PCs) can be handled as medical reference images as patient information, it will be very convenient.

For this purpose, if it is possible, for example, to convert a file format or the like (a storage format) of image data to a format compliant with an existing medical network standard (for example, the DICOM standard), it becomes possible to share data with medical workers such as doctors using a corresponding existing medical network.

Normally, an electronic image data file generated by an image acquisition apparatus such as a communication-function-equipped image pickup apparatus (such as a digital camera), an image-pickup/communication-function-equipped information terminal apparatus (such as a smartphone and a tablet PC) or the like in a common form is generally compliant with the Exif standard. The data format compliant with the Exif standard is a data format that is noncompliant with the DICOM standard.

Therefore, in the case of handling an object (an image data file) in a data format compliant with the Exif standard acquired using an image acquisition apparatus such as a communication-function-equipped image pickup apparatus (a digital camera or the like) or an image-pickup-communication-function-equipped terminal apparatus (a smartphone, a tablet PC or the like) in a common form as a medical object (a medical image data file) using a medical image observation apparatus compatible with the DICOM-compliant data format that is currently used widely in the medical field, there may be a case where accompanying information (metadata) accompanying the object (the image data file) cannot be accurately handled.

Note that, conventionally, a software application and the like for realizing cooperation among various kinds of systems and centralized management of image data using an apparatus compliant with the DICOM standard, for example, by performing an editing operation for an electronic image data file such as adding various kinds of information inputted by a hospital information system (HIS) and the like to accompanying information (metadata) of photographed image data, and converting image data in a DICOM-compliant data format to another data format (for example, a data format compliant with a picture archiving and communication system (PACS)).

### SUMMARY OF THE INVENTION

In common means for converting a data file, since it is necessary to perform conversion work after acquiring an image data file, the acquired image data file cannot be immediately used. Therefore, in such a system, there is a problem that it is not possible to efficiently use an image data file.

A medical image management system of one aspect of the present invention is a medical image management system creating, sharing and managing a medical image data file, the medical image management system including: an information terminal apparatus configured to transmit medical-related particular-use information about a particular individual; an image pickup apparatus configured to acquire an image data file; and a processor including hardware configured to associate medical service-related data obtained by receiving the medical-related particular-use information with the image data file to generate a medical image data file.

A medical image acquisition apparatus of one aspect of the present invention is provided with: a communication circuit configured to receive medical-related particular-use information; and a processor including hardware; and the processor associates image data acquired according to the medical-related particular-use information with service-related data obtained according to the medical-related particular-use information to generate a medical image data file.

A medical image data file acquisition method of one aspect of the present invention includes: transmitting medical-related particular-use information about a particular individual; receiving the medical-related particular-use information and performing an image pickup operation according to the medical-related particular-use information to acquire a general image data file in which image data and general metadata are associated with each other; and generating medical metadata based on the medical-related particular-use information and associating the medical metadata with the general image data file to generate a medical image data file.

A medical image management server of one aspect of the present invention is a medical image management server creating, sharing and managing a medical image data file, the medical image management server including a processor configured to perform communication control, wherein the processor configured to perform communication control receives, from an information terminal apparatus configured to transmit medical-related particular-use information about a particular individual, the medical-related particular-use information; instructs an image pickup apparatus to perform an image pickup operation according to the received medical-related particular-use information via communication; and acquires image data obtained by the image pickup operation performed by the image pickup apparatus.

Benefits of the present invention will be further apparent from detailed description below.

### BRIEF DESCRIPTION OF THE DRAWINGS

FIG. 1 is a conceptual diagram showing an outline configuration of a medical image management system of one embodiment of the present invention;

FIG. 2 is a conceptual diagram illustrating a first use pattern of operation of the medical image management system of the one embodiment of the present invention;

FIG. 3 is a conceptual diagram illustrating a second use pattern of the operation of the medical image management system of the one embodiment of the present invention;

FIG. 4 is a conceptual diagram illustrating a third use pattern of the operation of the medical image management system of the one embodiment of the present invention;

FIG. 5 is a block configuration diagram showing main components of each information terminal apparatus in the medical image management system of the one embodiment of the present invention;

FIG. 6 is a block configuration diagram showing main components of a file server in the medical image management system of the one embodiment of the present invention;

FIG. 7 is a diagram conceptually showing a configuration of an image data file generated in the medical image management system of the one embodiment of the present invention;

FIG. 8 is a conceptual diagram showing a state at the time of performing identity verification by a second terminal in the medical image management system of the one embodiment of the present invention;

FIG. 9 is a diagram showing a display example of “an identity verification menu” of the second terminal in the medical image management system of the one embodiment of the present invention;

FIG. 10 is a conceptual diagram conceptually showing flows of various kinds of information in the medical image management system of the one embodiment of the present invention;

FIG. 11 is a flowchart (a first half) showing operation of the second terminal among information terminal apparatuses in the medical image management system of the one embodiment of the present invention;

FIG. 12 is the flowchart (a latter half) showing the operation of the second terminal among the information terminal apparatuses in the medical image management system of the one embodiment of the present invention;

FIG. 13 is the flowchart showing operation of a first terminal among the information terminal apparatuses in the medical image management system of the one embodiment of the present invention;

FIG. 14 is a flowchart (a first half) showing operation of the file server in the medical image management system of the one embodiment of the present invention; and

FIG. 15 is the flowchart (a latter half) showing the operation of the file server in the medical image management system of the one embodiment of the present invention.

### DETAILED DESCRIPTION OF THE PREFERRED EMBODIMENT(S)

The present invention will be described below by an embodiment shown by drawings. Each drawing used in the description below is schematic, and a dimensional relationship, reduced scales and the like of respective members may be shown different for each component in order to show the component in a recognizable size on the drawing. Therefore, as for the quantity of each component, the shape of each component, a ratio of sizes among respective components, relative positional relationships among the respective components and the like described in each of the drawings, the present invention is not limited to a form shown in the drawing.

### One Embodiment

A medical image management system of one embodiment of the present invention is an image management system performing sharing and management of medical-related information, especially medical image data files (medical objects) by a network used in a medical facility such as a hospital (an existing external network or an in-hospital network), information terminal apparatuses and image acquisition apparatuses, and is an example of a network system in which, by making it possible to treat an object (an image data file) in a data format that is not compliant with the DICOM standard (hereinafter referred to as a data format noncompliant with DICOM) acquired by a generally widespread image acquisition apparatus in a normal form (for example, a communication-function-equipped image pickup apparatus such as a digital camera or an image-pickup/communication-function-equipped information terminal apparatus such as a smartphone or a tablet PC; hereinafter the apparatuses will be generically referred to as general image acquisition apparatuses) as a medical object (a medical image data file), effective use and management of a medical image data file is facilitated.

In assuming such an environment, in the embodiment of the present invention, in order that an image management system, an image acquisition apparatus and an image acquisition method capable of causing an image photographed or acquired by a general image acquisition apparatus to satisfy, for example, conditions required by medical image standards, for example, conditions of a doctor's instructions (including patient information) and resultant images, and a medical management data set can be provided, it is enabled to give order information, which is a big difference between a general image and a medical image (medical-related particular-use information about a particular individual including information indicating being an image based on the above doctor's instructions).

Since a medical image data file (a medical object) constitutes a medical management data set, it is preferable that a general image acquisition apparatus creating an image file enabling creation of a medical management data set includes, in addition to an image pickup portion configured to pick up an image of image data constituting a first part of the medical management data set, a communication portion configured to acquire order information (medical-related particular-use information about a particular individual and the like) constituting a second part of the medical management data set from outside, and a recording controlling portion configured to record the first and second parts of the medical image data set as a file.

The particular-use information is information for, at the time of performing photographing on an assumption that photographed image data is used for some purpose, identifying the purpose or use. In other words, the particular-use information is information including information about use of photographed image data and how the image data is expected to be treated when the image data obtained by photographing is outputted outside from an image pickup apparatus that has performed the photographing. Therefore, in the particular-use information, conditions at the time of obtaining particular image data are specified.

Especially, when “medical-related particular-use information” is written, a case is assumed where a doctor or a medical worker is required to photograph a state of a particular patient and acquire medical image data to be referred to for diagnosis, treatment and prescription. Since such information is often information brought from means other than an image pickup apparatus, the medical image acquisition apparatus (an image pickup apparatus) described in the present application includes a communication circuit configured to receive the above medical-related particular-use information from an external apparatus and the like.

Note that the communication circuit (which is not limited to a circuit specialized in communication but may be another circuit having a communication function) is also used at the time of, in order to handle acquired image data by another apparatus after photographing a target object using the image pickup apparatus, transmitting the image data from the image pickup apparatus to the outside (an information terminal or the like handled by a person who considers diagnosis, treatment and prescription using the image data).

Further, the “medical-related particular-use information” may include specifications at the time of transmitting image data (so to speak, “service information” such as to which kind of apparatus the image data is to be transmitted, in which form the image data is to be recorded, in which form the image data is to be displayed, and in which form the image data is to be printed).

By the service information being included in the medical-related particular-use information as described above, a person who transmits the “medical-related particular-use information” to the image pickup apparatus can request expected photographing. Further, when receiving image data as a result of the photographing, the person can receive, record, display and print the image data in an intended form based on the service information included in the medical-related particular-use information.

If the “medical-related particular-use information” in such a form is recorded as metadata in an image data file including image data as necessary, it becomes possible to record and transmit/receive a background that the image data had to be photographed, an expectation or intention of use of the image data and the like in association with the image data.

Photographing conditions at the time of acquiring image data may be included in “medical-related particular-use information”. Especially, in the case of a medical image, clarity of peripheral information such as information about who has requested photographing and information about who (or what) has been photographed is required. Therefore, it is preferable that such peripheral information is also included in the same image data file as metadata.

A control circuit for enabling such control that an image data file can be acquired according to the “medical-related particular-use information” obtained via the above communication circuit performs sequential control and operation according to necessary information at the time of photographing. In addition to such control, the control circuit performs exposure control and focusing control performed in a general camera, and may further have a function of confirming and identifying a target object.

The above control circuit associates image data acquired as described above with service-related data included in medical-related particular-use information acquired via communication with the outside by the above communication circuit to generate a desired medical image data file.

By including information about whether photographing based on medical-related particular-use information could be performed into a medical image data file obtained by such photographing, the medical image data file has evidence information about whether an obtained image is an image satisfying requirements and becomes image information that is useful for various judgments.

In other words, in the present invention, it is possible to cause standards for an image of a common terminal apparatus to satisfy, for example, conditions required by standards for a medical image.

More specifically, the medical image management system of the present embodiment is a network system that realizes data sharing of medical-related information such as patient information including image data as information to be supplied for particular use (for medical use), among image data acquired using “general image apparatuses (*2)” (that is, image data of “medical images (*3)”, “medical reference images (*4)” and the like) in addition to a function realized in a conventional network system (that is, a function of data sharing of medical-related information such as patient information including image data of “medical images (*3)” and the like acquired using “medical image acquisition apparatuses (*1)”), to facilitate management of medical image data.

### (*1 “Medical Image Acquisition Apparatuses”)

Here, the “medical image acquisition apparatuses” are apparatuses for image acquisition configured to be specialized in being used at a site in a particular field (here, a medical field). More specifically, for example, apparatuses such as an X-ray photographing apparatus (including a CT photographing apparatus and an angiographic photographing apparatus), an MRI apparatus, an ultrasonic tomographic photographing apparatus, an endoscope apparatus, a thermographic apparatus and a microscope photographing apparatus apply.

The medical image acquisition apparatuses are installed mainly in medical field facilities (for example, hospitals) and intended to be used only by medical-related qualified workers (referring to so-called medical staff such as doctors, nurses and various kinds of professional technicians; hereinafter referred to as medical workers) to acquire “medical images (*3)” (to be described later).

In general, the medical image acquisition apparatuses are not assumed to be used by persons other than medical workers including doctors. Therefore, all of image data files acquired by the above medical image acquisition apparatuses are approved as “medical images (*3)” (to be described later) and can be treated as medical objects (medical image data files), for example, in a data format compliant with the DICOM standard.

### (*2 “General Image Acquisition Apparatuses”)

The “general image acquisition apparatuses” are image acquisition apparatuses other than conventional “medical image acquisition apparatuses” and are assumed to be image acquisition apparatuses in a normal form that are generally widespread (for example, communication-function equipped image pickup apparatuses such as digital cameras, and image-pickup-function-equipped information terminal apparatuses such as smartphones and tablet PCs).

In this case, the “general image acquisition apparatuses” are assumed to be those which are used in:
(1) a case of being permanently provided in medical facilities and used by medical workers belonging to the medical facilities; and
(2) a case of being owned by persons other than medical workers (for example, caregivers such as patients' family members and relatives, or the patients themselves; hereinafter, such persons will be referred to as non-medical workers) and used by the non-medical workers such as the owners and the relatives.

Therefore, as images acquired by “general image acquisition apparatuses”, there are “medical images (*3)” (to be described later) acquired by medical workers using the apparatuses and “medical reference images (*4)” (to be described later) acquired by non-medical workers using the apparatuses.

### (*3 “Medical Images”)

The “medical images” are image information acquired by medical workers including doctors using predetermined medical image acquisition apparatuses according to “medical order information (*5)” issued by doctors as medical workers. The medical images are image information that can be approved as a part of patient information handled in the medical image management system of the present embodiment. Each medical image is in a form of an image data file formed including an image data body showing, for example, an affected part or a symptom as an image and “accompanying information (metadata) (*7)” accompanying the image data body.

More specifically, as a medical image data file handled in the system of the present embodiment, for example, a form of a medical object (a medical image data file) in a data format compliant with the DICOM standard is assumed.

More specifically, as situations in which the medical image is acquired, for example, the following situations are conceivable:
(1) a case where a doctor as a medical worker acquires an image using a predetermined medical image acquisition apparatus himself/herself based on his/her own medical care policies (information corresponding to “medical order information (*5)” (to be described later)) (examples: an endoscopic image, an ultrasonic tomographic image);
(2) a case where a doctor as a medical worker acquires an image using a predetermined medical image acquisition apparatus according to “medical order information (*5)” (to be described later) by another doctor (examples: an endoscopic image, an ultrasonic tomographic image); and
(3) a case where a medical worker (mainly a professional technician or the like) other than a doctor acquires an image using a predetermined medical image acquisition apparatus according to “medical order information (*5)” (to be described later) by a doctor as a medical worker (examples: an X-ray image, a CT image, an MRI image).

### (*4 “Medical Reference Images”)

The “medical reference images” refer to image information acquired by general medical workers who are medical workers other than doctors and professional technicians without particular qualifications to handle medical image acquisition apparatuses (for example, nurses and caseworkers; hereinafter referred to as general medical staff) and non-medical workers using “general image acquisition apparatuses” according to “medical order information (*5)” (to be described later) or “general order information (*6)” received from doctors.

The medical reference images are image information for reference with reliability enough to be recognized as patient information handled in the medical image management system of the present embodiment. The medical reference images are information referred to by doctors together with regular patient information including medical images when the doctors perform a medical action.

Since the medical reference images are images acquired by general medical staff and non-medical workers as described above, the medical reference images are not approved as “medical images” as formal patient information. However, the medical reference images are image information that can be recognized to be sufficiently reliable as information to be referred to when a doctor conducts a medical action such as medical care, and, therefore, the images are called medical reference images including the word “reference”.

Image information about a medical reference image is formed including an image data body representing, for example, an affected part or a symptom as an image and “accompanying information (metadata) (*7)” (to be described later) accompanying the image data body.

### (*5 “Medical Order Information”)

The “medical order information” is, for example, as shown below. In a normal case, after giving medical examination to a patient targeted by treatment, medical care, examination or the like (hereinafter referred to as a target patient), a doctor accesses a file server installed in a medical facility such as a hospital using a terminal apparatus in the hospital (for example, a terminal apparatus such as PC installed in a medical examination room in the hospital (a medical facility)) via an in-hospital network, reads out an electronic medical record file of the target patient from an electronic medical record database accumulated in the file server, and records the medical examination given on the day to the electronic medical record file. At that time, the doctor may also give one or more instructions such as an instruction of the next examination at the same time.

In order to give the instructions, the doctor opens a predetermined order information instruction menu screen using the terminal apparatus in the hospital and creates various kinds of instruction information corresponding to a desired examination or the like from the order information instruction menu screen. In this case, a group of pieces of instruction information including instructions of specific work as a plurality of medical actions corresponding to the particular examination or the like for the particular target patient is referred to as “medical order information”.

In the medical order information, for example, selection/specification of a desired photographing item from among a plurality of photographing classifications (for example, X-ray photographing, CT, MRI, endoscopic examination, ultrasonic examination and general photographing) and selection/specification of a desired photographing site (for example, a chest, an abdomen, a head or an elbow joint) according to the target patient are performed. In addition, a specific photographing instruction, for example, specification of the front and side of a left knee is given. Furthermore, if more detailed instructions are to be given as necessary, the detailed instructions are written, for example, in a comment field.

Here, an example of the detailed instructions in the medical order information is as follows.

For example, in the case of “medical order information for photographing images to observe the development of a wound of an affected part” about a predetermined target patient, the detailed instructions are:
(1) photographing is to be performed in a macro mode (photographing magnification 1:2);
(2) a predetermined color chart is to be included to perform photographing so that skin color can be judged; and the like.

It is also conceivable that not only an instruction to photograph a still image but also, for example, an instruction to photograph movie or an instruction to photograph a still image or movie including voice is given.

The medical order information created as described above is associated with patient information (a patient ID and the like) about the target patient and transferred to a terminal apparatus installed in a corresponding department for handling the examination and used by a medical worker or the like belonging to the department.

Here, medical order information at a medical site is commonly formed, including various kinds of information on “when”, “where”, “who” performs “which kind of examination”, “for whom”, “with which kind of apparatus”.

Information about “which kind of examination” may include information indicating photographing parameters and a photographing environment. In other words, the system or the apparatus of the present application may include a processor for classifying various kinds of information about an examination, such as information on “when”, “where”, “who”, “for whom”, “with which kind of apparatus”, “which kind of examination” and “under which photographing conditions”, from the medical-related particular-use information described above and generating a medical image data file including at least one of the above various kinds of information.

Such classification can be realized by an apparatus transmitting order information and an apparatus receiving the order information sharing information about what is indicated by which data, based on standards and the like, using a particular format and rules. Supplementary data for supplementarily describing, for each data, what kind of data the data is may be used so that each data group can be classified.

Since it is possible to perform such classification, the processor can make it easy to refer to only necessary data and check deficiency and inconsistency of data.

It is not necessarily required to classify all such information. It is important to classify necessary information and make it possible to judge whether there is deficiency or inconsistency. In other words, by including a processor configured to judge whether a predetermined item actually corresponds to or is inconsistent with a situation or a target object at the time of photographing, and whether information to be obtained at the time of photographing has been correctly obtained, recorded and associated with an image, in the system, it is possible to judge whether photographed image data meets a requested quality and required specifications.

In other words, by the processor including a function of judging whether it is possible to perform photographing based on the medical-related particular-use information described above, at the time of photographing, it is possible to prevent failure such as making a mistake about a target object to be photographed or about conditions such as a photographing environment.

As for “when” described above, for example, date and time information such as year, month, day and time applies. The date and time information can be obtained using a clock function that a photographing apparatus has.

As for “where” described above, information about a place where an apparatus and equipment corresponding to an examination to be performed are installed, such as an inside of a hospital and a related medical facility applies. The place information can be obtained using a position detection function that a photographing apparatus has.

As for “who” described above, a medical care department or a person in charge of the examination in a hospital applies. The information may be information specifying a department/person in charge of the examination.

As for “for whom” described above, such various kinds of information about a target patient as is written in an electronic medical record, for example, patient's basic attribute information about the target patient such as birthday, sex and blood type, and a patient ID apply. As for whether the information (the target patient information) corresponds to a target person facing an image pickup apparatus at the time of photographing, it can be confirmed using a face detection function or a voice judgment function of the image pickup apparatus.

For each of the above items, necessary information can be appropriately included at the time of photographing. If there is an inconsistency between instruction information and actual information about a target object at the time of photographing, it is possible to issue a warning, invalidate the data, record information to the effect that there is an inconsistency, to an image data file, and so on.

Such information is issued by an in-hospital system, a doctor or the like. However, when attention is paid to handling of personal information, it is not necessary to include all the information into medical order information. Only information of minimum necessary items may be included.

As for “which kind of apparatus” described above, an X-ray photographing apparatus, a CT apparatus, an MM apparatus, an endoscope apparatus, an image pickup apparatus (a camera), an information terminal apparatus (such as a smartphone) and the like apply. In the embodiment described here, however, it is assumed that a general camera, a smartphone or the like is used as the “apparatus”. It is an important point of attention of the present invention that such general apparatus can be treated as a medical apparatus as necessary.

As for “which kind of examination”, it is information about within which range, how many images are to be acquired for a site (for example, a chest or a leg) of the target patient.

More specifically, for example, in the case of performing general photographing of an external appearance of a predetermined affected part using an image pickup apparatus, an information terminal apparatus or the like, various kinds of photographing parameters such as composition at the time of photographing an image, a distance to the affected part, a focal length (an angle of view), photographing magnification, exposure, focus, aperture, shutter speed and with/without auxiliary light, image quality (resolution) setting, image size setting, file format setting (compressed image, uncompressed image or the like), setting of the number of images to be photographed, and the like apply. Note that the above information may be included in the information of the item of “with which kind of apparatus”.

As described above, the medical order information includes various pieces of information. In the medical order information, a photographing procedure or the like may be included, in which photographing is prompted with a display and the like of the photographing procedure so that the photographing is sequentially performed according to an ordering list or the like in which the medical order information is summarized as a list, whereby an examination and treatment can be performed according to the ordering list. In this case, there is an advantage of prevention of forgetting to perform photographing and enhancement of efficiency.

Note that the above patient basic attribute information can also be called “supplementary information for management”, “supplementary information for medical care”, “medical care management information” or the like.

The system and apparatus of the present application may include a processor capable of classifying various kinds of information such as the “supplementary information for management”, “supplementary information for medical care”, “medical care management information” and the like from the medical-related particular-use information described above.

Such classification can be realized by an apparatus transmitting order information and an apparatus receiving the order information sharing information about what is indicated by which data, based on standards and the like, using a particular format and rules. Supplementary data for supplementarily describing, for each data, what kind of data the data is may be used so that each data group can be classified. Since it is possible to perform such classification, the processor can make it easy to refer to only necessary data and check deficiency and inconsistency of data.

For example, even for the same target patient, a study may be different for each of symptoms of diseases, wounds and the like, and an examination (treatment including photographing) in a different situation may be required each time. Therefore, it becomes necessary to identify such a study and situation each time. For example, in the case of performing health examination every year, it is conceivable to handle study information as different study information from one another.

In the embodiment described below, information as described above is collectively described as medical order information. However, all the information is not necessarily required as necessary items each time for each situation. Only a part of the information required according to a corresponding situation can be appropriately adopted.

Thus, a group of pieces of information in which one or more medical action instructions are collected will be referred to as medical order information. The medical order information is created by a doctor, and it is only a medical worker including a doctor that executes the instructions included in the medical order information in response to the medical order information.

In short, the medical order information is a group of pieces of instruction information including one or more instructions of specific work as medical actions that is created by a doctor as a medical worker and issued from the doctor to a medical worker (mainly a professional technician or the like) including another doctor.

### (*6 “General Order Information”)

The “general order information” is a group of pieces of information issued mainly to a non-medical worker, in a group of pieces of information created by a doctor as a medical worker to be provided for particular use (more specifically, a group of pieces of information created by collecting one or more medical action instructions). The general order information is a group of pieces of particular-use information having content equivalent to content of the above medical order information but having a different destination address.

The general order information is a group of pieces of information created by a doctor, and content of the information may not be different from the above medical order information at all. For example, order information issued by a doctor may include an instruction that can be executed without special skills or expertise and without using special apparatuses. More specifically, for example, an instruction to take a general external appearance picture can be executed relatively easily even by a general non-medical worker.

A group of pieces of information including such an instruction issued by a doctor to a general non-medical worker is referred to as the general order information.

In general, even if a general non-medical worker receives the above medical order information, content of the medical order information is difficult for the general non-medical worker to implement (in terms of apparatuses, expertise and qualifications) in most cases. On the other hand, since a destination target of general order information is intended to be a general non-medical worker from the beginning, content of an instruction is limited.

Therefore, for example, a person who receives general order information and executes an instruction may be not a general non-medical worker but a medical worker, for example, a doctor or a professional technician. In the case where a medical worker such as a doctor receives general order information and executes an instruction, the general order information may be called medical order information. Information (for example, an image) acquired in this case can also be treated as medical information (image).

In short, general order information and medical order information are different in an assumed main receiver. Since the assumed receiver is different, content of the information necessarily differs according to the receiver.

### (*7 “Accompanying Information”)

The “accompanying information” described above is accompanying information that accompanies various kinds of image data, that is, metadata. The accompanying information is integrally recorded in a predetermined area of an image data file. Accompanying information of image data of a medical image or a medical reference image handled in the present invention is configured to include, in addition to basic information (for example, apparatus setting information, image format information and the like at the time of photographing) about an image data body, individual information of an image data file (patient information, a targeted case, photographing condition information set according to an affected part, and the like). Note that the image data file is configured with the image data body and the accompanying information, and the image data body and the accompanying information are integrated and exist in a form of one image data file as described above. As another form, a form in which the accompanying information is recorded as a separate file related to the image data file is also possible.

As a use example of an information terminal apparatus in the medical image management system of the present embodiment outside a medical facility, remote medical care, visiting medical care or home medical care can be assumed. In such cases, it is conceivable that an action of photographing an affected part or the like of a home patient to acquire image data is performed as one of medical actions in order to record a symptom.

For example, in the case of home medical care in which a patient stays at a place other than a medical facility, such as the patient's home, the situation is often a situation in which necessary medical apparatuses cannot be sufficiently prepared. This is because most of medical apparatuses are non-portable large-size apparatuses in a form of being fixed and used in a medical facility or the like. Even in the case of portable small-size medical apparatuses, since medical apparatuses are dedicated apparatus, the use of which is limited in most cases, it is difficult for a doctor or medical staff member to always carry many kinds of medical apparatuses.

Therefore, in order to support home medical care, it is considered to use, for example, consumer products that are generally widespread (general image acquisition apparatuses, for example, a communication-function-equipped image pickup apparatus such as a digital camera, and an information terminal apparatus such as an image-pickup-function-equipped smartphone and tablet PC). This is because, in recent years, this kind of general image-pickup/communication-function-equipped information terminal apparatus has been commonly owned and always carried by a patient, the patients' family members and the like.

Therefore, as a quasi-medical action, at the time of attempting to perform acquisition of a patient image using such an information terminal apparatus, if information included in the above medical order information or the above general order information can be used, it becomes possible to ensure reliability of acquired image data even if a photographer is a non-medical worker.

Further, if an image acquisition apparatus such as a general image-pickup/communication-function-equipped information terminal apparatus as described above is provided in a medical facility such as a hospital, a medical worker such as a doctor or a nurse can carry and use the image acquisition apparatus when visiting a patient's home (at the time of medical care visiting). In this case, since it is the medical worker that operates the information terminal apparatus, reliability of an acquired image is sufficiently secured.

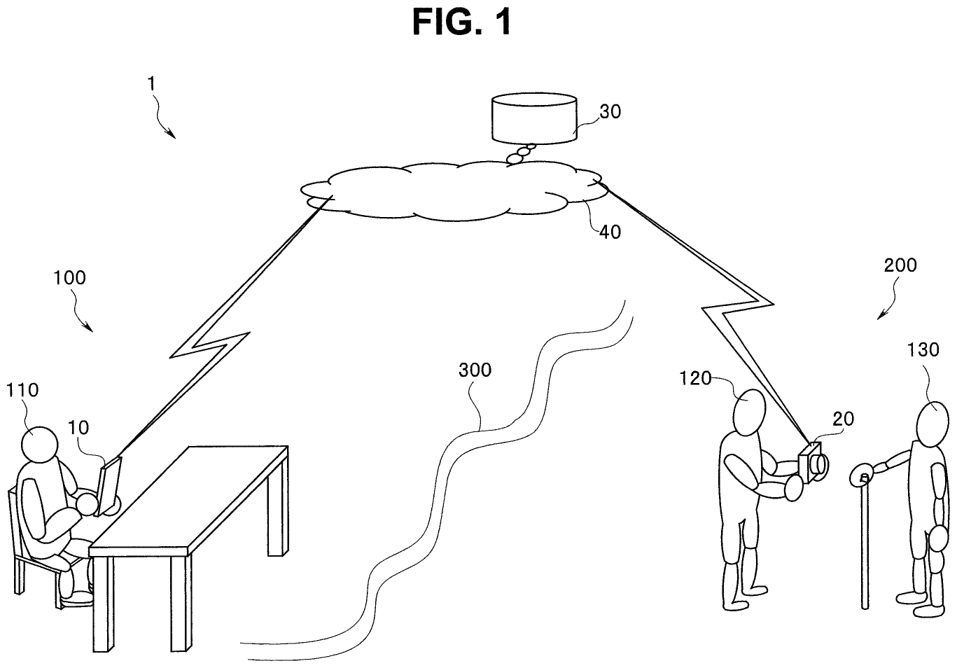

A concept of a medical image management system of one embodiment of the present invention assuming such a situation will be described below with reference to FIG. 1. FIG. 1 is a conceptual diagram showing an outline configuration of the medical image management system of the one embodiment of the present invention.

As shown in FIG. 1, a medical image management system 1 of the present embodiment is configured mainly with a plurality of information terminal apparatuses 10 and 20, a file server 30, a network 40 provided to connect the information terminal apparatuses 10 and 20 and the file server 30 to one another.

In this case, each of the plurality of information terminal apparatuses 10 and 20 is configured to be capable of mutually performing direct communication and capable of performing data communication with the file server 30 via the network 40.

As direct communication means between the plurality of information terminal apparatuses 10 and 20, for example, existing short-distance wireless communication means (Bluetooth (registered trademark)), wired communication means (mutual communication via cable connection) or the like is applied.

As the network 40 for performing communication among the plurality of information terminal apparatuses 10 and 20, and the file server 30, for example, an existing general network such as the Internet, a dedicated network using a dedicated line, or the like can be applied.

The file server 30 corresponds to, for example, a file server included in an existing hospital information system (HIS) or the like. In other words, the file server 30 is a data accumulation apparatus which is installed in a medical facility, for example, a hospital and in which various kinds of medical information including patient information and the like are accumulated. The file server 30 is connected to the network 40 including an in-hospital network in the hospital and external networks such as the Internet.

The plurality of information terminal apparatuses 10 and 20 are electronic apparatuses each of which is provided with an image pickup function capable of acquiring image information and a function of communication via the network 40 in addition to a function of direct communication between the respective information terminal apparatuses 10 and 20, and each of which is configured small and portable.

As a specific form of the plurality of information terminal apparatuses 10 and 20, for example, a communication-function-equipped image pickup apparatus (for example, a digital camera) or an image-pickup-function-equipped communication apparatus (for example, a smartphone and a tablet PC) is assumed.

In the example shown in FIG. 1, the information terminal apparatus indicated by reference numeral 10 between the plurality of information terminal apparatuses 10 and 20 is assumed to be an information terminal apparatus used by a person indicated by reference numeral 110 (for example, a doctor as a medical worker; hereinafter referred to as a doctor 110). In the description below, the information terminal apparatus is assumed to be a first information terminal apparatus and is referred to as a first terminal 10. In FIG. 1, the first terminal 10 is shown in a form of a tablet PC as an example.

The information terminal apparatus indicated by reference numeral 20 between the plurality of information terminal apparatuses 10 and 20 is assumed to be an information terminal apparatus used by a person indicated by reference numeral 120 (for example, a person who is a non-medical worker such as a patient's family member or a person taking care of a patient; of course, a medical worker is possible; hereinafter referred to as a patient's family member or the like 120 for easy understanding). In the description below, the information terminal apparatus is assumed to be a second information terminal apparatus and is referred to as a second terminal 20. In FIG. 1, the second terminal 20 is shown in a form of a communication-function-equipped digital camera, which is a general image pickup apparatus, as an example.

The second terminal 20 is a general image pickup apparatus which is privately owned by the patient's family member or the like 120 who is a non-medical worker and with which general photographing can be enjoyed as a normal camera. Under a special situation, a worthwhile function can be activated. In other words, it is not necessary to separately prepare a special apparatus; there is no burden of expenses and an installation place; and it is not necessary to borrow a special apparatus. In other words, it is devised that an apparatus usually used is useful for medical use as necessary.

Here, each of the plurality of information terminal apparatuses 10 and 20 may be in a form of a tablet PC or in a form of an image pickup apparatus (a digital camera or the like). Other forms such as a form of a smartphone are also possible. The plurality of information terminal apparatuses 10 and 20 have basically similar functions no matter which forms they are in.

In other words, as for information terminal apparatuses (10 and 20) applicable to the medical image management system 1 of the present embodiment, any apparatus can be similarly applied without being limited to the form the apparatus if the apparatus is equipped with at least an image pickup function and a communication function. Therefore, the forms of the respective information terminal apparatuses 10 and 20 shown in FIG. 1 are mere examples.

Note that, in FIG. 1, reference numeral 130 indicates a patient who is targeted by medical care. In FIG. 1, reference numeral 100 indicates a medical facility, such as a hospital, as a place where the doctor 110 exists. Reference numeral 200 indicates a home as a place where the patient's family member or the like 120 and the patient 130 exist.

Here, in FIG. 1, wavy lines indicated by reference numeral 300 indicate that the medical facility 100 and the home 200 exist at places away from each other.

An outline of operation of the medical image management system 1 of the present embodiment configured as described above is as follows.

First, it is assumed that, for example, in the home 200 or the like, the patient 130 living a normal life and the patient's family member or the like 120 exist. It is assumed that the doctor 110 belonging to the medical facility 100 is a home doctor of the patient 130. Therefore, it is assumed that patient information and the like about the patient 130 are accumulated in the file server 30 installed in the medical facility 100. It is assumed that the home 200 of the patient 130 is located away from the medical facility 100.

It is assumed that, in such a situation, for example, the patient's family member or the like 120 notices something unusual on the patient 130. If the patient's family member or the like 120 can photograph an image of an unusual part (for example, an affected part) of the patient 130 using the second terminal 20, which is a general image pickup apparatus (a digital camera) the patient's family member or the like 120 owns, and provide the image to the doctor 110 as a medical reference image, it will be very convenient.

However, the second terminal 20 is a general apparatus that is privately owned by an individual, and, as a matter of course, the second terminal 20 is not approved as a medical image acquisition apparatus. Therefore, in a normal case, an image acquired using such a general apparatus is not approved as a medical reference image.

Therefore, in the medical image management system 1 of the present invention, an image acquired using such a general apparatus (the second terminal 20) is configured so that the image can be approved as a medical reference image by the doctor 110 and can be treated, for example, as a medical object (a medical image data file) in a data format compliant with the DICOM standard.

As a procedure for the above, the patient's family member or the like 120 performs a procedure as shown below. In the situation as described above, the patient's family member or the like 120 sends request information to the effect that, since he/she wants to provide an image of the patient 130 photographed with the second terminal 20 he/she owns as a medical reference image, he/she will respond to an order if the doctor 110 issues the order, to the doctor 110. The part expressed as “request information” can also be expressed as “consultation information to doctor” or “inquiry information to doctor”. From a viewpoint of the spirit of the present application, since the patient's family member or the like 120 is requesting an order from the doctor 110, content of the “request information” is “order request information”. However, the simplified expression of “request information” is used here.

The request information is information including, for example, a matter to be consulted in terms of medical care for the patient 130 and recent situation information and the like about the patient 130. The patient's family member or the like 120 can send desired request information using the second terminal 20 to the first terminal 10 of the doctor 110 as necessary. This is because a person close to the patient 130 may feel some change in the patient 130 facing the patient 130 and because, even in such a case, the person cannot always take the patient 130 to the doctor 110 for medical care, or the patient 130 may refuse the medical care when the patient 130 is not conscious about obvious symptoms.

In such a situation, notification of something unusual about the patient 130, consultation about whether an image of the patient 130 is to be transmitted, and the like are specifically conceivable. It is conceivable that the doctor 110 requests some specific additional information about something unusual, and the doctor 110 may request deficient information and additional information in that case from the patient's family member or the like 120. Since the doctor 110 “requests” the information, the request will be described below as “order information”.

Note that since the “order information” issued from the doctor 110 is exemplified as being addressed to the patient's family member or the like 120, who is usually a non-medical worker, the “order information” corresponds to “general order information” as described above.

Since it is assumed that the second terminal 20 and the first terminal 10 are in an environment in which both are far away from each other in the situation described here (FIG. 1), communication between the second terminal 20 and the first terminal 10 is performed via the network 40 as shown in FIG. 1. Note that, if the second terminal 20 and the first terminal 10 are in a close range and can directly communicate with each other though the situation is not shown, the second terminal 20 and the first terminal 10 may perform direct communication by wireless communication or wired communication.

Note that, at the time of sending request information to the first terminal 10 using the second terminal 20, for example, an operation as below is performed.

First, the patient's family member or the like 120 creates request information that includes desired information to be transmitted to the doctor 110. Creation of the request information is basically performed on the second terminal 20. Since many information terminals are provided with a function capable of accepting a predetermined input operation to create a data file including arbitrary information, the function can be used if the second terminal 20 is one of information terminals in various forms such as a smartphone and a tablet terminal.

If the second terminal 20 is a general image pickup apparatus such as a digital camera or the like, an information inputting member and the like for creating arbitrary information data are normally not provided. In such a case, for example, the patient's family member or the like 120 can perform an operation of creating request information using a smartphone, a tablet PC or the like separately owned by the patient's family member or the like 120, transferring the created request information to an image pickup apparatus such as a digital camera used as the second terminal 20, and transmitting the request information to the first terminal 10 using a communication function of the image pickup apparatus.

Aside from the above, such means is also conceivable that application software compatible with the image pickup apparatus as the second terminal 20, for example, “a medical reference image acquisition application” or the like, which is application software for handling a medical reference image, is prepared and used.

In this case, a predetermined “medical reference image acquisition application” is installed in the image pickup apparatus, such as a digital camera, as the second terminal 20 in advance. The application software is program software capable of causing the image pickup apparatus such as a digital camera to operate in operation modes specialized in particular uses, for example, a request information creation mode, a request information transmission mode, a general order information reception mode, a medical reference image photographing mode and a medical reference image transfer mode. Therefore, it becomes possible thereby to create request information using only the image pickup apparatus, such as a digital camera, as the second terminal 20.

In this case, for example, the image pickup apparatus, such as a digital camera, as the second terminal 20 is caused to operate in the request information creation mode. In the request information creation mode, for example, a request information list included in the application (a list of assumed items of request information, or the like) is displayed, and a user creates desired request information by an operation of selecting a desired item using a touch panel or the like.

Here, the following is the reason why an apparatus of a category of an image pickup apparatus such as a digital camera is especially taken up as the second terminal 20.

An image pickup apparatus such as a digital camera is equipped with various functions specialized in acquisition of image data, that is, photographing, and is capable of easily changing photographing parameters such as composition, exposure, focus and color, and a degree of freedom of setting an angle of view and a photographing distance is high. Therefore, it is possible to respond to various requests (which may be included in order information) from the doctor 110. Further, the image pickup apparatus such as a digital camera is good at performing photographing by flashing strobe light or illuminating LED light and is capable of acquiring particular color information with special light. Furthermore, photographing of movie often includes information required by the doctor 110, and photographing of movie is also a function that the image pickup apparatus such as a digital camera is good at. The image pickup apparatus such as a digital camera is also equipped with a microphone function capable of recording voice, heart sound, breath sound and the like. Of course, when only such voice information is required, only a recording apparatus such as an IC recorder may be used as the second terminal 20.

The request information created in this way can be transmitted from the second terminal 20 to the first terminal 10 via direct communication or via the file server 30.

When the request information from the second terminal 20 arrives at the first terminal 10 of the doctor 110, the doctor 110 issues general order information according to the received request information in response to the request information. The information is “general order information” because the information is acquired by a general person other than a medical worker with a general apparatus.

In this case, it is assumed that “a general order information creation application”, which is application software specially designed to respond to a request from a general person lacking in medical knowledge, is installed in the first terminal 10 in advance. The general order information creation application includes a general order information creation mode in which guidance is given so that a non-expert person can easily acquire meaningful information, a general order information transmission mode devised so that information for having a general person respond to such technical needs is transmitted to (or received by) a so-called consumer apparatus owned by the general person and is correctly communicated to the counterpart, and the like as operation modes of the first terminal 10. For example, in order to perform exchange by telephone, e-mail or SNS (social networking service), reception is also required. Therefore, the general order information transmission mode may be expressed as a general order information communication mode.

When receiving the “request information”, the doctor 110 considers necessity to request additional information, and operates the first terminal 10 to communicate whether the additional information is necessary or not to the request information sender. In such a case, since the counterpart may not agree with the doctor, it is important to perform exchange (the general order information communication mode or a general order information transmission/reception mode). For example, subtle nuance is not communicated only by text-based information such as an e-mail, and it becomes impossible to make a correct judgment or a response depending on whether sentences are good or not. Therefore, there is a possibility that a necessary order cannot be issued.

At this time, in a case where the doctor 110 judges that more accurate information or additional information is required, it is necessary that the information is information that can be acquired by a general person with a general consumer apparatus. Therefore, the general order information creation application designed in consideration of the above is activated, and a predetermined operation is performed to create general order information (the general order information creation mode). Then, the created general order information is transmitted to the second terminal 20 (the general order information transmission mode).

The order here should not be such an order that requires a special qualification, a special apparatus and the like for an X-ray examination or the like. Simple information about such an auxiliary examination or a reference examination that substitutes for the X-ray examination or the like is to be ordered. Therefore, it is assumed that an operation is performed on a user interface (UI) with such icon design and menu configuration that only such an order can be selected.

Since the patient's family member or the like 120 is often not a medical-related person, description is made on the assumption that the patient's family member or the like 120 is a general person. The patient's family member or the like 120 receives the general order information for general persons that has been sent from the doctor 110 using the second terminal 20 (the general order information reception mode). At this time, it is necessary that medical terms are replaced with general words that even a person other than a medical worker can understand. If not, a non-medical worker (the patient's family member or the like) does not know what to do even if order information written with medical technical terms is issued and received by the non-medical worker.

As for the order information by the doctor 110, the “general order information creation application” is devisedly designed so that only simple orders, such as an order to verify identity of the patient 130, an order to take a picture of a particular part of the patient 130, and the like, can be issued. In other words, the “general order information creation application” is adapted so that a difficult order cannot be issued from the “general order information creation application”. When the doctor 110 issues a difficult order, the “general order information creation application” converts the order to such a form that even a non-medical worker can respond by replacing difficult words (medical technical terms and the like) issued by the doctor 110 with general easy words or converting a difficult procedure to a simple procedure. If it is difficult to do so, the “general order information creation application” does not issue the order or communicates an order to the effect that the patient's family member or the like 120 should leave the order of the doctor 110 to a specialist without doing anything, as an intention of the doctor 110.

As an example of simplification of work, order can be devised so that some settings are to be gradually made. For example, as a device for causing a particular site on a whole body image to be recognized, order at the time of sequentially photographing the whole body image and partially enlarged images may be included in order information.

As an example of a specific utilization technique, artificial intelligence and the like are given. The “general order information creation application” described above includes a function of, even if the doctor 110 uses a medical term, converting the medical term to a common language. More specifically, a database for term conversion, an inference engine by artificial intelligence or the like is adopted. The database, the inference engine or the like may exist on either the first or second terminal or may exist on the network connecting the first and second terminals.

Of course, an apparatus such as an image pickup apparatus such as a digital camera may be provided with such artificial intelligence and the database function. Since devises as described above are made, a request (an order) of the doctor 110 is replaced with such that everyone can respond to or a simple instruction that can be immediately understood by a general person, such as an advice to the effect that the request (the order) is to be left to a specialist and displayed or communicated by voice or the like. Therefore, it becomes possible for the patient's family member or the like 120 to respond to the order using or referring to such guidance and the like and perform desired photographing of the patient 130 using the second terminal 20 (the medical reference image photographing mode).

Thus, in an image data file that the patient's family member or the like 120 has acquired using the second terminal 20 in the situation shown in FIG. 1, that is, in response to a request (general order information) of the doctor 110, an image data body and various kinds of information included in the corresponding general order information as an accompanying information (metadata) accompanying the image data body are recorded (details will be described later). Consequently, the image data file is created as an image data file provided with a predetermined format (for example, a data format compliant with the DICOM standard) required to be approved as a medical reference image (details will be described later).

In other words, the patient's family member or the like 120 feels worried or anxious about a condition of the patient 130 and sends desired request information to the effect that the patient's family member or the like 120 wants to be told if there is anything he/she can do (information about apparatuses, instruments and the like that can be currently used may be additionally written here) to the doctor 110 in a manner of consulting the doctor 110 about which information is to be additionally sent. When receiving the request information, the doctor 110 considers the situation and issues appropriate general order information according to a judgment about the situation, it becomes possible for a general apparatus (the second terminal 20) that receives the general order information to acquire a medical reference image which is required by the doctor 110 and which is worth managing in the hospital or worth evaluating as medical information.

Then the medical reference image (which is not limited to an image but may be voice or other various kinds of information such as body temperature, weight and blood pressure) acquired in this way is recognized as such that responds to the order of the doctor 110. Furthermore, a judgment of the doctor 110 who receives the image is taken into consideration to secure a predetermined quality as a medical reference image.

It is preferable that there is accompanying information such as information about in which situation, which kind of information about which patient has been acquired by which kind of apparatus and in which way of using the apparatus. If what is included in the order information described above is used as some of such pieces of information, it will be efficient. The image data file is transferred from the second terminal 20 to the file server 30 or the like and accumulated as a part of patient information about the target patient 130.

Note that it is preferable that request information is such that a patient's family member or the like can input or select apparatuses, devices, systems, tools and the like that he/she can then so that a doctor appropriately issues an order based on a correct judgment.

For example, if there are a camera and a ruler, it is possible to apply the ruler to a wound and photograph an image including information about a size of the wound. If a communication system has a particular performance and is stable, diagnosis using a streaming image and diagnosis by a TV conference become possible.

Skills information and, in the case of sending a picture, information about a photographing technique acquisition level are also effective. The same instruction differs according to whether an order is given by an expression of “by decreasing the aperture” or by an expression of “by operating the dial of a camera”. Which expression is more understandable and enables work to be done more quickly is different according to a receiver who receives the instruction.

It is difficult for the doctor to determine which of such order candidates is to be selected unless sufficient judgment material is included in request information. The above request information may include information about a target patient and a user who uses an information terminal apparatus and may include information about apparatuses that the user using the information terminal apparatus can use. The above request information may include skills information about the user using the information terminal apparatus. The skills information may be any of information about ability to master and use apparatuses and tools (information terminal apparatuses may be included), information about sex, age and physical condition, information about language ability and writing ability, information about setting ability of communication and networks, and so on. There is a possibility that items that the doctor can request may differ according to the information.

As described before, in order that a general person can respond to a doctor's request (an order) using general apparatuses or tools, order information also must be written in simple expressions and communicated to a counterpart. For this purpose, it is required to utilize a graphic user interface accompanied by illustration, give a specific instruction showing an illumination condition or a distance condition, or dividing what could be very briefly explained to an expert into a plurality of steps and explaining each step or provide guidance information that is interactively implemented, or providing what could be very briefly explained to an expert as guidance information that is interactively shown, according to a situation or a difficulty level.

In other words, the above guidance information can be guidance information changed according to the request information. In other words, the present invention can be said to be a proposal of a patient information acquisition method for acquiring information about a target patient including: receiving request information including the information about the target patient and information about apparatuses that can be used by a user, creating guidance information according to the request information, and acquiring additional information acquired according to the guidance information, and can be said to be an invention of a system including an apparatus for achieving the method. The above is the expression without expressing a first terminal and a second terminal. However, a similar problem can be similarly solved by using the same apparatus. For example, such a modification as below is conceivable. When a patient's family member hands over a terminal to a doctor, a request is recorded in the terminal. Reading the request, after the doctor returns the same apparatus to the patient's family member, the patient's family member acquires information facing the patient, seeing guidance displayed on the terminal (according to an order).

For example, such a solution as above is conceivable for a patient's family member who can usually come to a hospital only in the daytime to consult a night shift doctor. In a case where a doctor is in a regular daily work shift, and a patient's symptoms appear at midnight, a similar solution method can be adopted. Such a method can be realized by providing a system, an apparatus and the like for realizing the idea.

Note that though FIG. 1 shows an example in which the patient's family member or the like 120 uses the second terminal 20, the present embodiment is not limited to the example. The second terminal 20 is similarly used in a situation of being used by the patient 130 himself/herself. The basic schematic operation of the present embodiment is as described above.

If such conditions are included in request information, there is a possibility that a judgment made by a doctor changes. For example, it is difficult for a patient himself/herself to photograph a picture of his/her back or the like. A judgment of a doctor changes according to whether there is a mirror (corresponding to one of the tools described above).

Next, operation of the medical image management system 1 of the present embodiment will be briefly described for each specific use situation (use pattern).

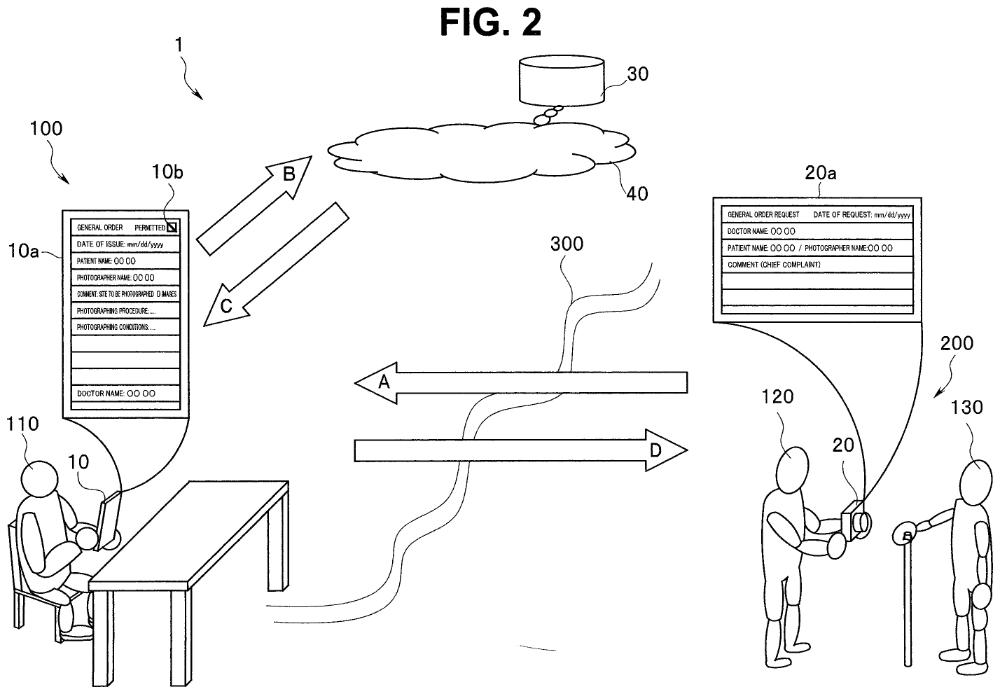

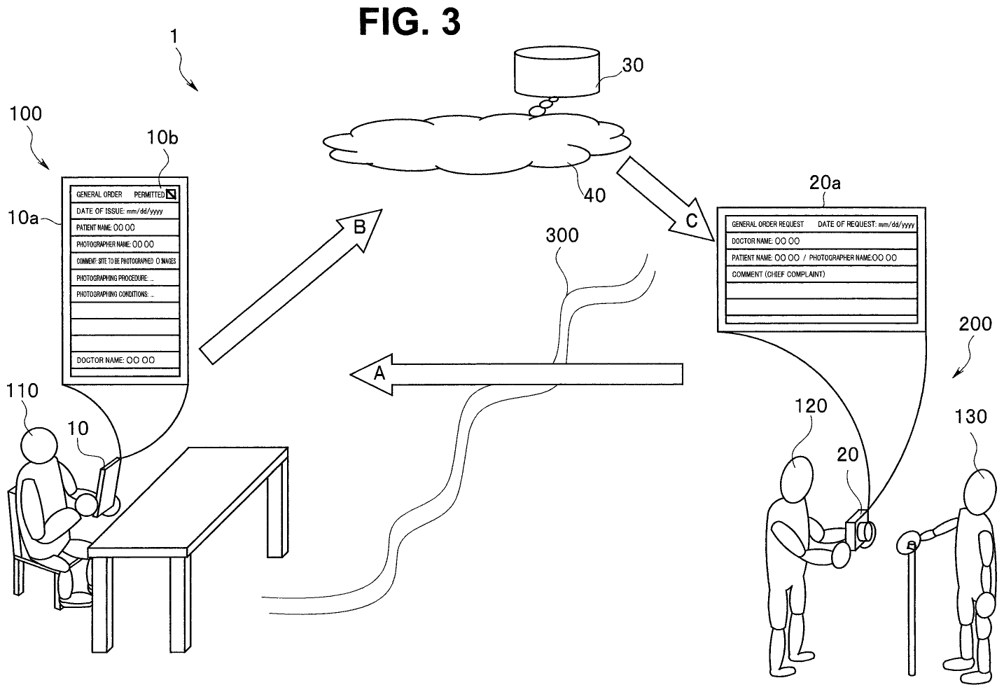

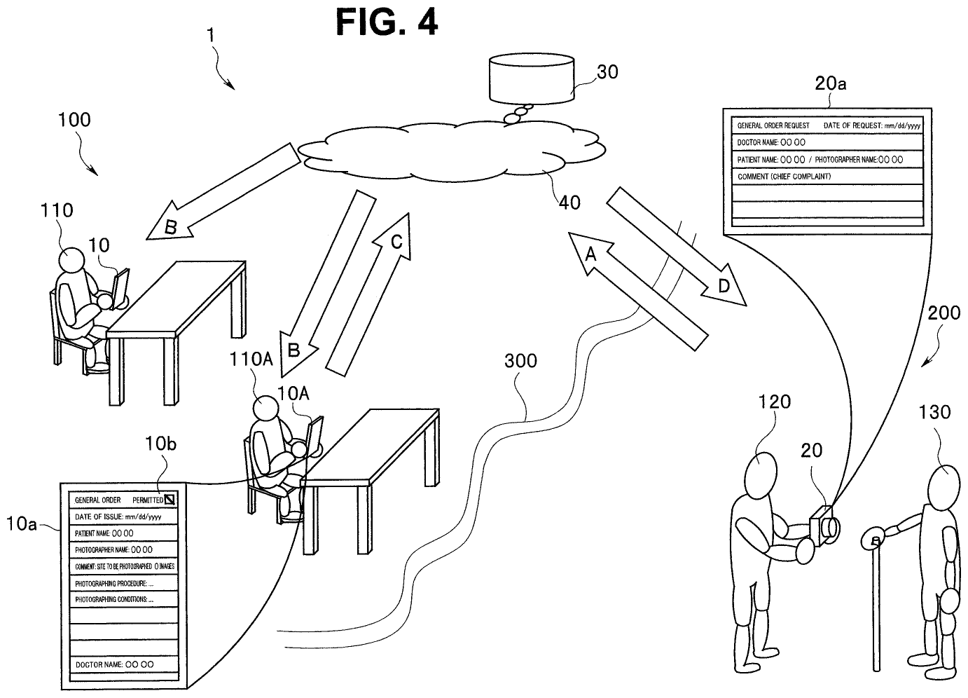

FIGS. 2 to 4 are conceptual diagrams showing an outline of the operation of the medical image management system of the present embodiment according to use situations. Among FIGS. 2 to 4, FIG. 2 is a conceptual diagram illustrating a first use pattern of the operation of the medical image management system of the present embodiment. FIG. 3 is a conceptual diagram illustrating a second use pattern of the operation of the medical image management system of the present embodiment. FIG. 4 is a conceptual diagram illustrating a third use pattern of the operation of the medical image management system of the present embodiment.

### ([First Use Pattern])

First, the first use pattern of a use situation of the medical image management system 1 of the present embodiment is as follows (see FIG. 2).

Here, the first pattern assumes a case where a relationship between a doctor as a home doctor of, for example, a clinic (a privately managed small-scale medical facility) and a patient (including the patient's family members) is relatively strong.

First, the patient's family member or the like 120 performs direct communication with the first terminal 10 of the doctor 110 to transmit request information using the second terminal 20 (arrow reference symbol A in FIG. 2: the request information transmission mode).

In this case, it is assumed that a terminal to receive the request information is the first terminal 10 of the doctor 110, but the terminal is not limited to the first terminal 10. For example, if it is the medical facility 100 that manages the first terminal 10, there may be a case where such a procedure occurs that a receptionist (a general medical worker, for example, a nurse or a qualified medical clerk) in the medical facility 100 performs a reception operation and then communicates the request information to the doctor 110, in addition to a case where the doctor 110 directly receives the request information using the first terminal 10 first. Here, for simplification of description, the doctor 110 is illustrated as a representative example of a receiver in the medical facility 100.

Note that even if the first terminal 10 receives the request information from the second terminal 20, the doctor 110, the receptionist or the like does not necessarily immediately (in real time) confirm the request information that has arrived at the first terminal 10. Therefore, after transmitting the request information from the second terminal 20, the second terminal 20 is in a state of waiting for a response from the first terminal 10.

Here, as content of the request information sent from the second terminal 20, there are, in addition to basic information such as a title of “General Order Request”, year, month and date of the request, a target doctor name, a patient name and a photographer name, chief complaint information and the like as comments, for example, as indicated by reference numeral 20a in FIG. 2. As the main complaint information, a reason for transmitting the current request information, a matter to be consulted in terms of medical care of the patient 130, recent situation information and the like about the patient 130, and the like can be specifically written.

The request information sent from the second terminal 20 is received by the first terminal 10. The doctor 110 confirms the received request information using the first terminal 10, for example, when he/she has time.

The doctor 110 who has confirmed the request information accesses the file server 30 using the first terminal 10 (arrow reference symbol B in FIG. 2) and refers to medical record data and the like about the target patient 130 of the received request information (arrow reference symbol C in FIG. 2).

The doctor 110 creates general order information according to the received request information using the first terminal 10. After that, the doctor 110 directly connects to the second terminal 20 using the first terminal 10 and sends the created general order information to the second terminal 20 (arrow reference symbol D in FIG. 2).

Here, as content of the general order information created on the first terminal 10, there are, in addition to basic information such as a title of “General Order”, issue year, month and date, the patient name, a patient ID (patient-specific identification information), the photographer name, and an issuer name (information about a doctor in charge and the medical institution including the name of the doctor in charge), various kinds of information (a site specified to be photographed, the number of images required, a photographing procedure, photographing conditions and the like) according to the chief complaint as comments, for example, like a display screen indicated by reference numeral 20a in FIG. 2.

Note that, here, the photographing conditions include, for example, a photographing format (a file format), various kinds of settings such as image aspect ratio information, and instruction information such as an instruction to include a scale in a screen. Furthermore, as the photographing condition information, setting information and the like about various kinds of photographing parameters and the like to enable an equal and similar image to be photographed at each time of photographing, and thumbnail images of sample images, and the like are also included.

Note that the above setting information includes various kinds of image pickup related information, for example, exposure information, white balance information, sensitivity information, optical system focal length information, information about a distance to an object, photographing magnification information and angle information.

Note that, in addition to medical metadata, various kinds of information (for example, a photographing place (GPS information), date and time information and photographing condition information) included in an image data file or the like having a data format compliant with the Exif standard, which is conventionally used for a common image file, are recorded as general metadata.

Furthermore, approval information for being approved as a medical reference image is attached to general order information. In the form example shown in FIG. 2, an icon display indicating “permitted” and a permission display 10b in a form of a check mark being inputted are shown.

When receiving such general order information (the general order information reception mode), the second terminal 20 displays content of the general order information on the display screen, for example, in a form equivalent to the form indicated by reference numeral 10a in FIG. 2 (note that display of reception by the second terminal 20 is not shown (see reference symbol 10a in FIG. 2)).

Then, the patient's family member or the like 120 as a photographer executes photographing of the patient 130 according to the received general order information using the second terminal 20.

The second terminal 20 may be provided with a predetermined photographing operation mode for, at the time of performing photographing according to general order information, automatically making various kinds of settings and the like so that an appropriate photographing operation can be easily executed. A photographer can perform appropriate photographing only by switching to the predetermined photographing operation mode. Here, as the predetermined photographing operation mode, for example, the “medical reference image photographing mode” or the like is assumed. As for the medical reference image photographing mode of the second terminal 20 in this case, for example, a corresponding application can also be installed in the second terminal 20 in advance.

To image data acquired in this way, various kinds of information included in the general order information is recorded as accompanying information (metadata) (to be described in detail later).

In other words, an image data file handled in the medical image management system 1 of the present embodiment is configured such that medical metadata including medical information included in order information or general order information is recorded to common metadata (referred to as general metadata), which is conventionally included in a general image data file (for example, Exif (exchangeable image file format) data) as accompanying information.

In other words, an image data file acquired by the second terminal 20 (the image acquisition apparatus used by the patient's family member or the like 120) among information terminal apparatuses is in a data format that is conventionally generally widespread, and, more specifically, for example, a data format compliant with Exif (exchangeable image file format).

In the medical image management system 1 of the present embodiment, an image data file (an Exif file) acquired by the second terminal 20 is recorded in a form of being added with corresponding medical metadata. In this case, in the system of the present embodiment, a basic data format of an image data file generated in the end is a format which be easily converted, for example, to a data format compliant with the DICOM standard.

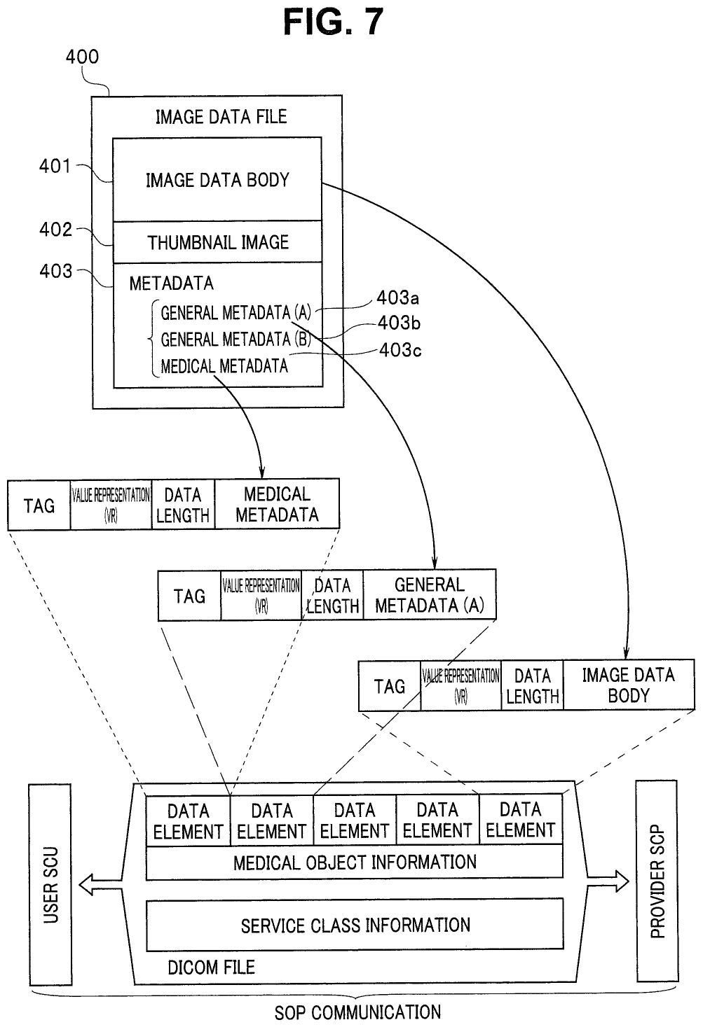

Here, FIG. 7 is a diagram conceptually showing a configuration of an image data file generated in the medical image management system of the present embodiment.

Normally, a general Exif image data file is an image data file configured with an image data body 401, a thumbnail image 402 and metadata 403 (see FIG. 7).

Here, the metadata 403 includes various kinds of common information, for example, date and time information, photographing apparatus information (a body name, a lens name and the like), and photographing condition information (in addition to setting information such as exposure, white balance, sensitivity and optical system focal length information, information about a relationship with an object such as information about a distance to the object, photographing magnification information and angle information). Hereinafter, the above general metadata will be referred to as general metadata.

In the medical image management system 1 of the present embodiment, such a configuration is made that, when a photographing operation based on general order information is performed using an information terminal apparatus (the second terminal 20), medical information included in the general order information is added to the metadata 403 and recorded to an acquired Exif image data file.

In other words, in the medical image management system 1 of the present embodiment, an image data file 400 with such a structure as shown in FIG. 7 is generated. The image data file 400 is an image data file configured with the image data body 401, the thumbnail image 402 and the metadata 403. Among the above, the metadata 403 includes general metadata (A) 403a, general metadata (B) 403b and medical metadata 403c.

By adopting such a data file configuration, it is possible to be easily compatible with SOP class communication. In the SOP communication, it is understandably standardized which processing (S: service) is assumed for each data (0: object). Since image data obtained by normal photographing is intended to be appreciated, information about “which service” is assumed is not especially required. A medical image or the like handled in the present application, however, is photographed with a clear intention or purpose (e.g., imaging of a particular patient, in a particular way, using particular imaging equipment, pursuant to a particular study requested by a particular physician). Therefore, if image data is associated with “particular-use information”, it is more understandable, and favorable to prevent confusion at the time of handling.

In other words, the particular-use information here may include information for, at the time of performing photographing on an assumption that photographed image data is used for some purpose, identifying the purpose or use of the image data. In other words, the particular-use information is information including information about use of photographed image data and how the image data is expected to be treated when the image data obtained by photographing is outputted outside from an image pickup apparatus that has performed the photographing. Therefore, in the particular-use information, conditions at the time of obtaining particular image data may be specified.

Especially, when “medical-related particular-use information” is specified, a case is assumed where a doctor or a medical worker is required to photograph a state of a particular patient and acquire medical image data to be referred to for diagnosis, treatment and prescription. Since such information is often information brought from means other than an image pickup apparatus, the medical image acquisition apparatus (an image pickup apparatus) described in the present application includes a communication circuit configured to receive the above medical-related particular-use information from an external apparatus and the like.

Note that the communication circuit (which is not limited to a circuit specialized in communication but may be another circuit having a communication function) is also used at the time of, in order to handle acquired image data by another apparatus after photographing a target object using the image pickup apparatus, transmitting the image data from the image pickup apparatus to the outside (an information terminal or the like handled by a person who considers diagnosis, treatment and prescription using the image data).

Further, the “medical-related particular-use information” may include specifications at the time of transmitting image data (so to speak, “service information” such as to which kind of apparatus the image data is to be transmitted, in which form the image data is to be recorded, in which form the image data is to be displayed, and in which form the image data is to be printed).

By the service information being included in the medical-related particular-use information as described above, a person who transmits the “medical-related particular-use information” to the image pickup apparatus can request expected photographing. Further, when receiving image data as a result of the photographing, the person can receive, record, display and print the image data in an intended form based on the service information included in the medical-related particular-use information.

If the “medical-related particular-use information” in such a form is recorded as metadata in an image data file including image data as necessary, it becomes possible to record and transmit/receive a background that the image data had to be photographed, an expectation or intention of use of the image data and the like in association with the image data.

Photographing conditions at the time of acquiring image data may be included in “medical-related particular-use information”. Especially, in the case of a medical image, clarity of peripheral information such as information about who has requested photographing and information about who (or what) has been photographed is required. Therefore, it is preferable that such peripheral information is also included in the same image data file as metadata.

A control circuit for enabling such control that an image data file can be acquired according to the “medical-related particular-use information” obtained via the above communication circuit performs sequential control and operation according to necessary information at the time of photographing. In addition to such control, the control circuit performs exposure control and focusing control performed in a general camera, and may further have a function of confirming and identifying a target object.

The above control circuit associates image data acquired as described above with service-related data included in medical-related particular-use information acquired via communication with the outside by the above communication circuit to generate a desired medical image data file.

In other words, the medical image acquisition apparatus here is characterized in including a communication circuit configured to receive medical-related particular-use information. Furthermore, in the medical image acquisition apparatus, a controlling portion such as a processor associates image data acquired according to the medical-related particular-use information with service-related data obtained according to the medical-related particular-use information to generate a medical image data file. In other words, by the control circuit (the processor) including a function of judging whether it is possible to perform photographing based on the medical-related particular-use information described above, at the time of photographing, it is possible to prevent failure such as making a mistake about a target object or about conditions such as a photographing environment.

As should be appreciated from the foregoing, the medical-related particular-use information may include information to be used before the image is captured (e.g., the patient to be imaged, the person or organization to perform the imaging, the imaging equipment to be used, how the imaging equipment is to be used, settings to be used by the imaging equipment, required imaging conditions, etc.) and/or information to be used after the image is captured and to be associated with the image (e.g., any of the above information, and/or a study to which the imaging belongs, a physician who ordered the study, etc.)

Here, it is assumed that the general metadata (A) 403a refers to such general-use information that is included in data in a data format compliant with the DICOM standard (for example, date and time information, photographing apparatus information and photographing condition information). It is assumed that the general metadata (B) 403b refers to such information that is not included in data in a data format compatible with the DICOM standard (for example, GPS information) among metadata included in data in a data format compliant with Exif. The medical metadata 403c is a group of pieces of information in which medical information included in general order information is taken.

Each of data components included in the image data file 400, that is, each of the image data body 401, the thumbnail image 402, the general metadata (A) 403a, the general metadata (B) 403b and the medical metadata 403c has a data structure configured with a data element tag, a value representation (VR), a data length and a data body as shown in FIG. 7.

The data element tag is a symbol showing information for expressing what the data element indicates. Details of the data element tag are specified in the DICOM standard. More specifically, for example, Study Date, Study Time, Patient's Name, Patient's ID and the like are specified.

A value representation (VR) is information showing which kind of data the data element is. The value representation (VR) is a 2-byte character string, and details are specified in the DICOM standard. More specifically, for example, there are AS (age string), AT (attribute tag), CS (code string), DA (date), DT (date/time), LT (long text), PN (person name), ST (short text), TM (time), UI (unique identifier) and the like.

The data length is the number of bytes of the data body.

The data body is an area where target data is stored.

Note that since a DICOM file is configured with a plurality of data elements and service type information, it becomes possible to, if each of the data elements constituting the image data file 400 is configured as described above, easily perform conversion to medical object information in a data format compliant with the DICOM standard.

The above is a configuration for clarifying a purpose of data, that is, for what the data is, as described later (see description about SOP class communication), and the configuration can be said to be a way of performing communication in which data and information about how the data is to be handled are combined.

A DICOM file refers to what is obtained by storing DICOM data attached with a preamble, as a file. However, “DICOM data” includes not only a DICOM file but also a format of a packet internally used in DICOM communication, and the like. What is shown here is expressed as a file because importance is attached to content of what is sent. However, actually, a communication format may be included because the communication format can also be standardized and determined; and “file” may also be referred to as “medical management data set”.

In other words, a DICOM file is configured with a plurality of data elements and service type information as described above.

The service type information is information showing to which functions (service types) stored data elements respond.

As service types, for example, there are a communication management function (Verification Service Class), an inter-apparatus image transfer function (Storage Service Class), a function of inquiring about/searching for various kinds of medical information and transferring an image (Query Retrieve Service Class), an image printing function (Print Management Service Class) and the like.

At the time of performing communication of a data file compliant with the DICOM standard, an SOP (service-object pair class) class communication function is used in which a service user (SCU: service class user) on a client terminal side requests establishment of communication, and a service provider (SCP: service class provider) on a server side responds to the request.

This is communication for clarifying what (object) and how (service) a user wants to do. Therefore, a DICOM file is in a data format in which service (handling and the like; a function) type information and a plurality of pieces of data element information (such as data; objects) are combined as a set. In other words, SOP class communication can be said to be a way of performing communication in which data and information about how the data is to be handled are combined.

In the present embodiment, in order to realize such DICOM standard communication, an image data file acquired by an image acquisition apparatus is adapted to have data elements corresponding to object information.

Note that the metadata 403 included in an image data file may further include, in addition to the general metadata (A) 403a, the general metadata (B) 403b and the medical metadata 403c, third metadata including data of a result of confirmation about whether acquired image data has been obtained by photographing according to order information (to be described later).

Furthermore, additionally, a secure area which is a predetermined area is provided, and important information requiring confidentiality (various kinds of information including personal information and the like) including the medical metadata 403c and the like may be recorded in the secure area.

In this case, special encryption processing is performed for the information recorded in the secure area so that the information can be read only by an apparatus included in a particular system for which security countermeasures are taken (for example, an in-hospital system). By doing so, it is not possible to, even if a medical image data file handled in the present embodiment is, for example, copied to a portable recording medium or leaked outside via a network, read the information in the secure area using an apparatus not compatible with the medical image data file. Therefore, there is an advantage that it is possible to easily perform security management.

Note that the medical metadata 403c may be configured to exist in a form of a file separate from the conventional general metadata 403a and 403b or may be integrally configured as shown in FIG. 7 described above. However, no matter which form is adopted, it is important that the image data body 401 and the medical metadata 403c are associated with each other. Since the image data body 401 and the medical metadata 403c are associated with each other, the associated metadata 403 necessarily accompanies when a predetermined operation is performed, at the time of performing an operation on the image data body 401 (for example, an operation for movement to another medium, copying or deletion) even if each of the image data body 401 and the medical metadata 403c exists as a single data file. Therefore, for example, it does not happen that only the medical metadata 403c exists alone.

In the metadata 403, in addition to the medical metadata 403c constituted by information included in general order information inputted by the doctor 110 as described above, for example, items overlapping with existing general metadata, for example, date and time information and photographing apparatus information (such as camera-specific information) at the time of photographing are recorded as general metadata (A) 403a.

If it is not for the first time that the first terminal 10 receives request information from the second terminal 20 of the particular patient's family member or the like 120, but there are use results in the past, then request information received before should already exist in the first terminal 10.

Therefore, when the first terminal 10 receives request information from the second terminal 20 as described above, for example, corresponding patient information (for example, a patient ID) can be automatically detected. Therefore, using the patient information detected by the first terminal 10, the doctor 110 can automatically and quickly search for desired information in the file server 30.

Then, an image data file acquired using the second terminal 20 is transferred to the file server 30 by some means that is not shown (the medical reference image transfer mode).

For example, the image data file is transmitted from the second terminal 20 to the file server 30 via the network 40 and recorded to a predetermined area in the file server 30. Then, at this time, the image data file is associated with patient information about the patient 130 among a plurality of pieces of patient information recorded in the predetermined area of the file server 30.

The acquired image data file is recorded to the file server 30 via a reception department of the medical facility 100 when the patient's family member or the like 120 or the patient 130 visits the medical facility 100 carrying the second terminal 20 or provided to the first terminal 10 of the doctor 110.

Thus, by predetermined medical metadata 403c included in medical order information or general order information being given to an image data file acquired by the second terminal 20, the image data file is approved by the doctor 110, and can be treated as an image data file of a medical reference image as a medical object (a medical image data file), for example, in a data format compliant with the DICOM standard. Therefore, the doctor 110 can refer to the image data as a medical reference image for medical care.

Note that though it is described in the description above that various kinds of information related to photographing (photographing condition information) are included in the metadata 403, the present embodiment is not limited to the above. The photographing condition information may be created as a data file separate from an image data file. The data file of the photographing condition information is recorded in association with the corresponding image data file. Therefore, for example, when image data is transferred to another apparatus, a related photographing condition information data file is necessarily transferred together with the image data at the same time as a set.

When the patient's family member or the like 120 performs predetermined photographing of the patient 130 using the second terminal 20 that has received general order information, identification information for identifying the patient 130 may be recorded.

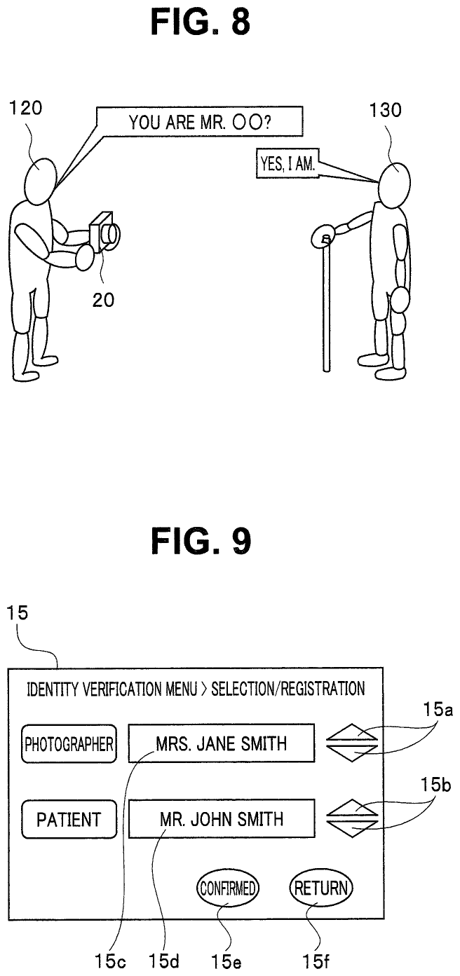

As for the identification information, for example, when the patient's family member or the like 120 performs photographing facing the patient 130 as shown in FIG. 8, the patient's family member or the like 120 performs identity verification of the patient 130 by calling the patient 130 to cause the patient 130 to give his/her name. For this purpose, the second terminal 20 is provided with a configuration for recording voice for the confirmation (a microphone and a voice recording portion). Voice data recorded then is associated with image data acquired after that. The voice data may be recorded being added to metadata of an acquired image data file.

In order to avoid impersonation by voice, video may be photographed to record a state of confirmation by conversation to be used as evidence. Since confirmation that should be originally done by a doctor or a medical worker in charge is entrusted to a terminal apparatus and an operator, preservation of evidence is important. Therefore, it is better to record which order by a doctor at which point of time the identity verification is based on. In the case of requiring further strictness, a doctor's terminal, and a communication line such as a telephone line or a network may be used only then to cause the doctor or a medical worker to directly talk with a patient and record the talk.

As for the identification information, a face recognition technique that the second terminal 20 is provided with can be used. Here, as the second terminal 20, an information terminal apparatus owned by a family member or a relative of the patient 130 or the patient 130 himself/herself is assumed. Therefore, a face image of the patient 130 himself/herself is registered with the second terminal 20 in advance. By performing photographing using the second terminal 20, it can be easily detected that the patient 130 himself/herself is targeted by photographing.

Furthermore, as for the identification information, the second terminal 20 may be configured being added with a fingerprint authentication technique, an iris recognition technique or the like to use the technique.

Furthermore, such a form is also conceivable that, for example, “an identity verification menu” or the like is provided in a menu setting mode of the second terminal 20 so that a photographer name (a name of a patient's family member or the like) and a patient name that are registered in advance can be selected and set (see FIG. 9).

In a display example shown in FIG. 9, a photographer name (a name of a patient's family member or the like) and a patient name that are registered in advance are sequentially displayed in display fields 15c and 15d, for example, by operating selection icons 15a and 15b. Then, by operating a CONFIRMED icon 15e when desired names are displayed in the display fields 15c and 15d, setting is completed. By operating a Return icon 15f after selecting and setting the photographer name (the name of the patient's family member or the like) and the patient name, it is possible to exit the setting menu and return to the photographing mode. Then, by executing a predetermined photographing operation in the photographing mode, the set name information is recorded as medical metadata.

When detecting the patient 130 himself/herself as described above, the second terminal 20 may judge that a medical reference image is to be photographed and perform control to automatically switch to a corresponding predetermined photographing operation mode (the medical reference image photographing mode).

When the second terminal 20 switches to the medical reference image photographing mode, a controlling portion (to be described later) of the second terminal 20 refers to photographing condition information and the like included in received general order information to perform control to automatically set photographing parameters (such as an exposure setting, white balance setting, sensitivity setting and focal length setting) that are optimal to acquire a new medical reference image. At this time, a guidance display (an assist display) of photographing condition information such as composition, a photographing angle and a distance to an object is automatically displayed in a display portion. As for the display, for example, a form of being superimposedly displayed on a live view image on the display portion is conceivable.

Note that though description has been made above with the patient's family member or the like 120 as a person who sends request information and is a photographer as an example, the present embodiment is not limited to the example. For example, when a situation of a doctor performing visiting medical care, visiting nursing care or the like is considered, a photographer facing the patient 130 may be a different person, but the present embodiment is similarly applicable. In that case, a nurse, a certified care worker or the like visiting the patient's home corresponds to the person who operates the second terminal 20.

In this way, it is possible to cause an image data file acquired by the patient's family member or the like 120 other than a general medical worker and a medical worker using a general information terminal apparatus (the second terminal 20) to be handled as a medical reference image approved by the doctor 110 and converted to a data format compliant with the DICOM standard.

The outline of the first use pattern of the use situation of the medical image management system 1 of the present embodiment is as described above.

### ([Second Use Pattern])

Next, the second use pattern of the use situation of the medical image management system 1 of the present embodiment is as follows (see FIG. 3). Here, basically, the second use pattern is substantially the same as the first use pattern. The second use pattern described below is different in a point that details of creation of general order information are not performed by a doctor but by automatic processing by the system.

It is the same as the first use pattern described above that the patient's family member or the like 120 creates request information using the second terminal 20 first (the request information creation mode) and performs direct communication with the first terminal 10 of the doctor 110 to transmit the request information (arrow reference symbol A in FIG. 3; the request information transmission mode). Content of the request information sent from the second terminal 20 at this time is also similar to the content described for the first use pattern above.

When confirming the request information received by the first terminal 10, the doctor 110 accesses the file server 30 using the first terminal 10 to issue predetermined instruction information corresponding to the target patient 130 of the received request information to the file server 30 (arrow reference symbol B in FIG. 3).

The instruction information issued from the doctor 110 here is only basic information including the request information, and a very simple instruction according to the patient 130.

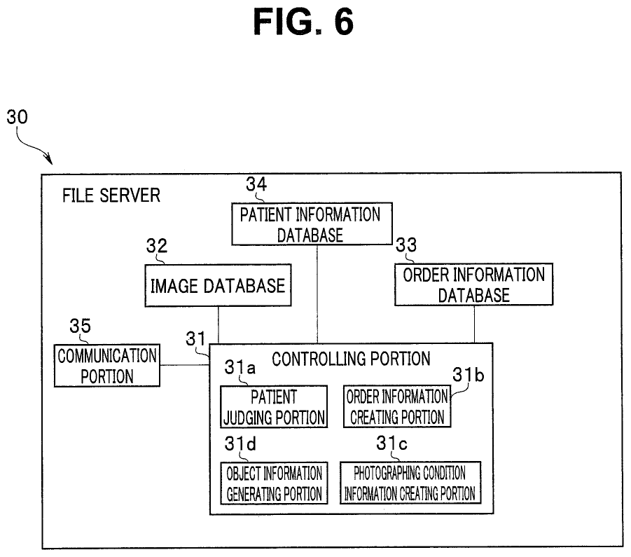

Receiving the instruction information of the doctor 110, a controlling portion 31 (to be described later; see FIG. 6) of the file server 30 creates general order information according to the request information. After that, the file server 30 sends the created general order information to the second terminal 20 (arrow reference symbol C in FIG. 3). The second terminal 20 receives the general order information (the general order information reception mode). After that, predetermined photographing is performed according to the received general order information (the medical reference image photographing mode). Then, image data, which is a photographing result, is transferred to the file server 30 (the medical reference image transfer mode).

Content of the general order information created by the file server 30 here is similar to the content described for the first use pattern above.

Operation of the second terminal 20 which has received the general order information is also similar to the operation in the case of the first use pattern.

Thus, the second use pattern is the same in the point that it is possible to cause image data acquired by the patient's family member or the like 120 other than a general medical worker and a medical worker using a general information terminal apparatus (the second terminal 20) to be handled as a medical reference image that can be approved by the doctor 110.

Further, in the second use pattern, by entrusting the general order information creation processing to automatic processing by the file server 30, it is possible to lessen a doctor's manual work and, therefore, reduce a burden on the doctor.

Note that though description is made on an assumption that the controlling portion 31 (see FIG. 6) of the file server 30 creates general order information according to request information in the description of the second use pattern above, the present embodiment is not limited to the example. For example, the instruction information from the doctor 110 may be transferred to an information processing apparatus such as a PC (not shown) installed in the reception department in the medical facility 100 via the file server 30 so that the general order information creation processing may be performed by the information processing apparatus of the reception department and a receptionist who handles the information processing apparatus.

The general order information created by the automatic processing or by the receptionist in the second use pattern may not be sent to the second terminal 20 immediately after being created but may be confirmed by the doctor 110 once before being sent after creation. By doing so, reliability of the general order information can be further enhanced.

The outline of the second use pattern of the use situation of the medical image management system 1 of the present embodiment is as described above.

### ([Third Use Pattern])

Next, the third use pattern of the use situation of the medical image management system 1 of the present embodiment is as follows (see FIG. 4). Here, basically, the third use pattern is substantially the same as the first use pattern and the like. The third use pattern described below assumes a case of being operated, for example, in a medical facility such as a large-scale hospital provided with a lot of doctors and a fully equipped hospital system.

In this kind of large-scale medical facility, work generally starts after passing through a reception system first. It is common that a team constituted by a plurality of doctors is systematically in charge of a particular patient. Therefore, a particular doctor does not always respond to a particular patient. Further, there are many patients that one doctor is in charge of

Therefore, if information related to a plurality of patients a particular doctor in charge of is directly transmitted to the doctor's first terminal 10, it is naturally impossible for the reception-side doctor to respond to all of the information. In the third use pattern, in consideration of the above, all request information sent from a patient side is accumulated in the file server 30 via the reception department of the hospital system first (arrow reference symbol A in FIG. 4).

When request information is inputted to the file server 30, the file server 30 refers to information included in the request information and simultaneously delivers the request information to a plurality of doctors in charge (arrow reference symbol B in FIG. 4).

Note that though two persons indicated by reference symbols 110 and 110A are shown as the plurality of doctors in charge in the example shown in FIG. 4, more doctors may exist.

The plurality of doctors in charge (110, 110A . . . ) confirm the request information at different timings, for example, because of difference in working hours or being engaged in other work (for example, performing an operation). Therefore, for example, one doctor (in the example shown in FIG. 4, a doctor indicated by reference symbol 110A) who can confirm the request information first, among the plurality of doctors in charge (110, 110A . . . ) to respond to a target patient of the request information, creates general order information.

Here, a procedure for the doctor 110A to create the general order information is similar to the procedure described above for the first use pattern. The general order information created by the doctor 110A in this way is transferred to the file server 30 and recorded in association with the target patient (arrow reference symbol C in FIG. 4).

At this time, when the general order information according to the request information is created and registered with the file server 30, the file server 30 sends status (situation state) information indicating “having been processed” to the other doctors to whom the relevant request information has been simultaneously delivered (in the example shown in FIG. 4, the doctor 110). It is possible, thereby, to prevent such duplicated processing that a plurality of pieces of general order information are created for a certain one piece of request information.

The general order information registered with the file server 30 is transmitted being addressed to a sender of the corresponding request information (the second terminal 20) after passing through the reception department.

According to such a configuration, it is also possible to similarly apply the present embodiment to a large-scale medical facility and obtain similar effects.

The outline of the third use pattern of the use situation of the medical image management system 1 of the present embodiment is as described above.

In order to realize the operation as described above, main components of the information terminal apparatuses (the first terminal 10 and the second terminal 20) and the file server 30 of the medical image management system 1 of the present embodiment will be described below.

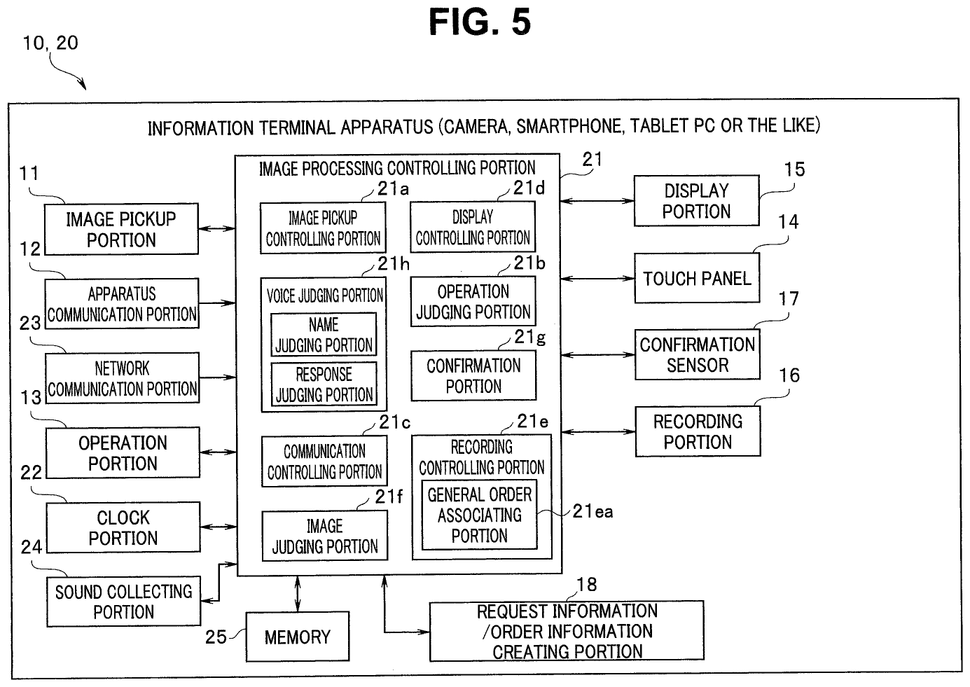

FIG. 5 is a block configuration diagram showing main components of each information terminal apparatus in the medical image management system of the present embodiment. FIG. 6 is a block configuration diagram showing main components of the file server in the medical image management system of the present embodiment.

First, the main components of the information terminal apparatuses (10 and 20) in the medical image management system 1 of the present embodiment will be described. As described above, the information terminal apparatuses are the first terminal 10 used by a doctor and the second terminal 20 used by a person other than a doctor (for example, a medical worker other than a doctor, or a patient's family member). The main components of the information terminal apparatuses are substantially the same even if forms of the information terminal apparatuses are different. Therefore, description will be made below on an assumption that the first terminal 10 and the second terminal 20 have the same components.

As shown in FIG. 5, the information terminal apparatus (10 or 20) is configured mainly being provided with an image pickup portion 11, an apparatus communication portion 12, a network communication portion 23, an operation portion 13, a touch panel 14, a display portion 15, a recording portion 16, a confirmation sensor 17, a sound collecting portion 24, a clock portion 22, an image processing controlling portion 21, a request information/order information creating portion 18, a memory 25 and the like.

Note that, as components of the information terminal apparatus (10 or 20), various components are provided in addition to the components enumerated here. However, since the components are not directly related to the spirit of the present invention, the components are not shown or described. It is assumed that, as for the various kinds of components that are not shown or described, components similar to components conventionally applied to an information terminal apparatus in a general form are provided.

The image pickup portion 11 is a component unit including an optical system configured to form an optical image and a circuit or the like including a photoelectric conversion element or the like configured to convert an optical object image formed by the optical system to an electrical signal, and the like, and configured to realize an image pickup function.

Note that, as for specific components of the image pickup portion 11, components that are substantially the same as components of a conventional image pickup unit in a general form are applied. The image pickup portion 11 is controlled by an image pickup controlling portion 21a to be described later.

The apparatus communication portion 12 is a component unit configured including a circuit unit (for example, short-distance wireless communication means) configured to realize communication performed between apparatuses.

The network communication portion 23 is a component unit configured including a circuit unit configured to realize communication with the file server 30 via the network 40.

For example, the network communication portion 23 receives medical-related particular-use information from outside and transmits a result of photographing (image data) to the outside as an image data file associated with the medical-related particular-use information. Of course, the network communication portion 23 may be a circuit or program capable of performing data communication with the outside, for example, by the SOP class communication described with reference to FIG. 7 in addition to the image data file transmission function.

Note that the communication means included in the apparatus communication portion 12 and the network communication portion 23 is not limited to wireless communication means, but wired communication means is also included. The apparatus communication portion 12 and the network communication portion 23 are controlled by a communication controlling portion 21c to be described later.

In the second terminal 20, the apparatus communication portion 12 and the network communication portion 23 (generically referred to as communication portions) have a transmission function of transmitting request information to the first terminal 10 or the file server 30 as well as a reception function of receiving general order information sent from the first terminal 10 or the file server 30.

The operation portion 13 is a component unit including an operation member and a circuit portion for, in response to various kinds of operations performed by a user, causing corresponding various kinds of instruction signals to be generated.

The touch panel 14 is an operation member arranged in a form of being superimposed on a display screen of the display portion 15. The touch panel 14 is an operation member configured to, in response to operation instructions by the user's finger or the like, cause corresponding various kinds of instruction signals to be generated.

Note that though the touch panel 14 is included in the operation portion 13, the touch panel 14 is shown separately from the operation portion 13 because the touch panel 14 is one of operation members often applied to various electronic apparatuses nowadays and is an especially important component.

Which of the operation portion 13 and the touch panel 14 has been operated is judged by an operation judging portion 21b to be described later. Based on a result of the judgment, the image processing controlling portion 21 outputs a corresponding predetermined operation signal to a corresponding component unit.

The display portion 15 is a component unit including a display panel, a driving circuit for the display panel and the like (not shown) and configured to realize a display function. On the display panel of the display portion 15, an image based on image data acquired using the image pickup portion 11 and menu images generated by various kinds of setting programs and the like are displayed. The display portion 15 is controlled by a display controlling portion 21d.

The recording portion 16 is a component unit for recording various kinds of data files. As the data files recorded to the recording portion 16, there are, for example, an image data file including image data and metadata (general metadata and medical metadata), setting information which is a file in which various kinds of settings such as settings specific to setting items required at the time of performing predetermined photographing are collected (more specifically, for example, setting information and the like in which various kinds of settings to be made at the time of acquiring a medical reference image are collected), apparatus information and the like about the information terminal apparatus (10 or 20) and communication counterparts' apparatuses (a terminal, a file server or the like) and other various kinds of information. To the recording portion 16, voice data acquired by the sound collecting portion 24 is also recorded.

The clock portion 22 is an internal clock circuit that is called a so-called real time clock (RTC). The clock portion 22 is a circuit portion used at the time of performing temporary control, for example, giving date and time information or time information to an image data file, measuring a time period between predetermined instruction timings during control processing.

The sound collecting portion 24 is a component unit including a microphone, a driving circuit for the microphone, and the like and is configured to collect voice data. The sound collecting portion 24 is controlled by the image processing controlling portion 21. Various kinds of judgments by a voice judging portion 21h are made based on the voice data acquired by the sound collecting portion 24. The voice data acquired by the sound collecting portion 24 is recorded to the recording portion 16.

The image processing controlling portion 21 is a control circuit configured to comprehensively control the information terminal apparatus (10 or 20) and is an image processing circuit configured to perform various kinds of image processing based on various kinds of image data. The image processing controlling portion 21 is configured with a processor including hardware.

The image processing controlling portion 21 is configured being provided with the image pickup controlling portion 21a, the operation judging portion 21b, the communication controlling portion 21c, the display controlling portion 21d, a recording controlling portion 21e, an image judging portion 21f, the voice judging portion 21h and the like.

The image processing controlling portion 21 corresponds to a processor of a medical image acquisition apparatus. For example, the image processing controlling portion 21 has a function of effectively utilizing medical-related particular-use information obtained by the above communication portion. More specifically, for example, the image processing controlling portion 21 has a function of, at the time of recording image data obtained according to the medical-related particular-use information, giving appropriate information among various kinds of information included in the medical-related particular-use information to acquired image data as metadata to create an image data file, and the like. Such control is performed mainly by the recording controlling portion 21e.

The image data file generated by the image processing controlling portion 21 is recorded in a form of holding necessary information. Therefore, when the recorded image data file is transmitted to another external apparatus, effective information related to image data is also transmitted together.

According to a system configuration, a form is also possible in which, for example, what is converted to an SOP level is transmitted instead of an image data file. As for conversion of a communication form in this case, the processor (for example, the communication controlling portion 21c or the like) can perform the conversion, appropriately referring to predetermined rules.

In other words, in addition to transmission of an image data file, a circuit or program capable of performing output to another external apparatus via such SOP class communication as illustrated in FIG. 7 can also be adopted.

The image processing controlling portion 21 (processor) controls how and which target object is to be photographed according to acquired medical-related particular-use information. In that case, for example, the image pickup controlling portion 21a controls, in addition to photographing parameters corresponding to brightness and a distance of a target object, photographing parameters (an angle of view, focusing, white balance, exposure, gradation correction, contrast correction, special image processing and the like) so that a photographed target object can be reproduced in a size and color that are appropriate for diagnosis and judgment.

As for judgment about whether a target object is a correct target object corresponding to medical-related particular-use information, the image judging portion 21f or the voice judging portion 21h can perform the judgment based on information about whether a target object image is an assumed one or whether a voice or response is a voice or response of a correct object. In this case, if it is judged that the image, voice or response is different from the assumed image, voice or response, control such as disabling an image pickup operation may be performed in cooperation with the image pickup controlling portion 21a, or a warning display may be given by a function of the display controlling portion 21d, or information to the effect that the photographing is not correct photographing may be recorded in cooperation with the recording controlling portion 21e. Alternatively, or in addition, the medical-related particular-use information may be used (directly or indirectly) to automatically set parameters of the image pickup apparatus. Alternatively, or in addition, the medical-related particular-use information may include or otherwise reference step-by-step guidance instructions to be followed by the person operating the image pickup apparatus to acquire image(s) of the patient.

Since there may be a case where photographing is difficult for a beginner to perform, a guidance display such as handling instructions may be given while a photographer is looking at a target object at the time of photographing so that required photographing can be performed. For example, an instruction about what to do next or a guidance display for preventing problems about access to and a composition of a target object may be given. Furthermore, an advice display may be given by comparing an ideal photographing result specified in advance and a photographing situation in which photographing is going to be performed, and judging a difference between the ideal photographing result and the photographing situation. More specifically, for example, in a dark environment, a guidance display to recommend photographing at a brighter place or a guidance display to recommend use of auxiliary light is given. An advice display to recommend use of a tripod to prevent a camera shake from occurring at the time of photographing may be given. Furthermore, in a dark environment, since a blurred image is obtained when a patient moves, advice information such as “Please stop” and “Please do not move” may be given by voice in addition to a guidance display. Note that, in order to handle voice information, it is necessary to further provide, for example, a voice reproduction circuit or a voice generation apparatus (such as a speaker).

The recording controlling portion 21e associates acquired image data with service-related data obtained according to the medical-related particular-use information to generate an image data file. It is possible, thereby, to cause information about for what reason image data has been photographed to be metadata to create an image data file. As information of the metadata, photographing conditions, photographing parameters and information about a photographing environment may be given. Here, by recording a photographing place and a date and time together, arrangement after photographing is facilitated, and evidence of a photographing result (image data) is enhanced.

Information about how image data is to be treated after photographing may be acquired and recorded. It is possible, thereby, to generate an image file in which an image is correctly picked up, and even use of the image file is correctly specified. Furthermore, processing for improving security, such as batch encryption processing of related image files and falsification prevention processing, may be performed as necessary.

As described above, the image pickup controlling portion 21a is a circuit portion or program software configured to control the image pickup portion 11 to acquire data for displaying a live view image and execute an image pickup operation of acquiring still image data or movie image data in response to a predetermined operation.

The operation judging portion 21b is a circuit portion or program software configured to, in response to an input from the operation portion 13, judge an inputted operation instruction signal. In response to a result of the judgment by the operation judging portion 21b, the image processing controlling portion 21 executes control of a corresponding component unit.

As described above, the communication controlling portion 21c is a circuit portion or program software configured to control the apparatus communication portion 12 and the network communication portion 23. In response to an instruction signal from the operation portion 13 or the like, the communication controlling portion 21c performs communication-related control. For example, the image processing controlling portion 21 makes a selection about whether communication is to be performed between terminal apparatuses or communication with the file server 30 is to be performed via the network 40 and controls the selected communication means to perform corresponding various kinds of communication controls.

It is reception of “particular-use information” that is important here, and the particular-use information is information for, when photographing is performed on an assumption that photographed image data is used for some purpose, identifying the purpose or use. In other words, the particular-use information is information including information about use of photographed image data and how the image data is expected to be treated when the image data is outputted outside from an image pickup apparatus that has performed the photographing. Therefore, in the particular-use information, conditions at the time of obtaining particular image data are specified.

Especially, when “medical-related particular-use information” is described, a case is assumed where a doctor or a medical worker is required to photograph a state of a particular patient and acquire medical image data to be referred to for diagnosis, treatment and prescription. Since such information is often information brought from an apparatus other than an image pickup apparatus, the medical image acquisition apparatus (an image pickup apparatus) described in the present application includes a communication circuit configured to receive the medical-related particular-use information from an external apparatus and the like.

Note that the communication circuit (which is not limited to a circuit specialized in communication but may be another circuit having a communication function) is also used at the time of transmitting image data acquired after photographing a target object using the image pickup apparatus to the outside (an information terminal or the like handled by a person who uses the image data to consider diagnosis, treatment and prescription) from the image pickup apparatus in order that the image data is handled by another apparatus.

The above control circuit associates image data acquired as described above and service-related data included in medical-related particular-use information acquired via communication with the outside by the above communication circuit with each other to generate a desired medical image data file.

The display controlling portion 21d is a circuit portion or program software configured to control the display portion 15. At the time of photographing, it is possible to display an image pickup result and confirm a composition at the time of framing. Of course, it is also possible to display a recorded image at the time of image reproduction. Medical-related particular-use information may be displayed, and metadata of a generated image file may be displayed.

In response to an instruction signal from the operation portion 13 or the like, the display controlling portion 21d performs display-related control, for example, performs control to select or switch display content to be displayed by the display panel of the display portion 15.

As described above, various warnings, advices and guidance may be displayed according to conditions, and countermeasures for security are taken by displaying settings at the time of communication so that a correct communication counterpart can be selected.

The recording controlling portion 21e is a circuit portion or program software configured to control the recording portion 16. In response to an instruction signal from the operation portion 13 or the like, the recording controlling portion 21e performs recording-related control, for example, performs control to convert various kinds of information to be recorded to the recording portion 16 to a predetermined data format and record the various kinds of information to a predetermined area of a predetermined storage medium (not shown).

The recording controlling portion 21e also has a general order associating portion 21ea. The general order associating portion 21ea is a component portion configured to play a role of treating information included in general order information created by a doctor or the order information creating portion 31b of the file server 30 as medical metadata; creating, by associating the medical metadata with an image data file acquired by the image pickup portion 11, one image data file as a medical object; and handing over the medical object to the recording portion 16.

In other words, the general order associating portion 21ea of the recording controlling portion 21e is a component portion configured to play a role of arranging image data, photographing-related information, order-related information and the like to make a file so that a medical management data set (see FIG. 7) can be created, and handing over the file to the recording portion 16.

Here, the general order associating portion 21ea is a component configured to clarify for what the data is. In other words, the general order associating portion 21ea may perform control for combination, rearrangement, rewriting and the like of data to cause the data to follow a way of communication in which data and information about how the data is to be handled are combined (SOP class communication). Since the SOP class communication is communication in which a purpose of data acquisition and information about use (service) are integrated, the image processing controlling portion 21 performs, according to particular-use information obtained by the communication portion, use of information according to the particular-use information.

As described before, the particular-use information here is information for, when photographing is performed on an assumption that photographed image data is used for some purpose, identifying the purpose or use. In other words, the particular-use information is information including information about use of photographed image data and how the image data is expected to be treated when the image data is outputted outside from an image pickup apparatus that has performed the photographing.

Further, the “medical-related particular-use information” may include specifications at the time of receiving image data (from where and to which image pickup apparatus a photographing request is made, and the like) and specifications at the time of transmitting image data (so to speak, “service information” such as to which kind of apparatus the image data is to be transmitted, in which form the image data is to be recorded, in which form the information is to be displayed, and in which form the information is to be printed).

By the service information being included in the medical-related particular-use information as described above, a person who transmits the “medical-related particular-use information” to the image pickup apparatus can request expected photographing. Further, when receiving image data as a result of the photographing, the person can receive, record, display and print the image data in an intended form based on the service information included in the medical-related particular-use information.

If the “medical-related particular-use information” in such a form is recorded as metadata in an image data file including image data as necessary, it becomes possible to record and transmit/receive a background that the image data had to be photographed, an expectation or intention of use of the image data and the like in association with the image data.

Photographing conditions at the time of acquiring image data may be included in “medical-related particular-use information”. Especially, in the case of a medical image, clarity of peripheral information such as information about who has requested photographing and information about who (or what) has been photographed is required. Therefore, it is preferable that such peripheral information is also included in the same image data file as metadata.

In order that such metadata can be generated, necessary information is prepared by the above “particular-use information”. Especially in the case of the “medical-related particular-use information”, information about articles related to medical care, and a patient's food, excrement and the like at the time of requesting photographing may be included in addition to information about “patient”, “affected part” and the like.

A control circuit for enabling such control that an image data file can be acquired according to the “medical-related particular-use information” obtained via the above communication circuit performs sequential control and operation according to necessary information at the time of photographing. In addition to such control, the control circuit performs exposure control and focusing control performed in a general camera, and may further have a function of confirming and identifying a target object.

The above control circuit associates image data acquired as described above with service-related data included in medical-related particular-use information acquired via communication with the outside by the above communication circuit to generate a desired medical image data file.

Note that information included in the medical metadata recorded to the recording portion 16 further includes, in addition to the various kinds of information enumerated in the above description, for example, information about communication (communication information), patient information (target information), information about a doctor, information about an order by the doctor (order information and request information), information about treatment and examination of an affected part (condition information), information about an apparatus (apparatus identification information (such as a GS1 Code, a JAN Code, and apparatus serial number) and the like), color chart information, measurement information, information about apparatus posture and position, approval information, information about whether real or false and the like as a medical image or a medical reference image, and other various kinds of information.

In the recording portion 16, general order information and the like created by a doctor or the order information creating portion 31b of the file server 30 and received by the information terminal apparatus (10 or 20) are also recorded.

The image judging portion 21f is a circuit portion or program software configured to perform judgment processing for various kinds of information acquired by analyzing, based on image data acquired by the image pickup portion 11, image data transferred from another terminal apparatus or the like and inputted, each piece of image data, for example, judgment processing for light source information about a photographing environment, information about a distance to an object, object (for example, face area) information and the like.

The voice judging portion 21h is a component portion configured to perform predetermined judgment processing based on voice data acquired by the sound collecting portion 24. Therefore, the voice judging portion 21h has processing circuits such as a name judging portion and a response judging portion. Here, for example, the name judging portion detects human voice in voice data acquired by the sound collecting portion 24 and, if human voice is detected, judges whether the human voice is data giving a name. Similarly, for example, the response judging portion detects human voice in voice data acquired by the sound collecting portion 24 and, if human voice is detected, judges whether the human voice is data giving a response.

More specifically, for example, at the time of performing photographing of a patient in the medical reference image photographing mode, the patient is called for identity verification before photographing, for example, immediately before photographing as described above. In that case, the name judgment and the response judgment are performed based on voice data acquired by the sound collecting portion 24.

A confirmation portion 21g is a circuit portion or program software configured to confirm whether or not an image data file acquired by the image pickup portion 11 has been obtained by photographing according to corresponding order information.

The confirmation sensor 17 is a sensor member, an electronic circuit or the like for acquiring related information required at the time of performing confirmation by the confirmation portion 21g based on an acquired image data file. As the confirmation sensor 17, there is, for example, a sensor member, an electronic circuit or the like configured to realize a face recognition technique, a fingerprint authentication technique, an iris authentication technique and the like as techniques for performing identity verification.

The request information/order information creating portion 18 is a circuit portion or program software configured to create order information in response to an input from the operation portion 13 when the information terminal apparatus is used as the first terminal 10 and create request information in response to an input from the operation portion 13 when the information terminal apparatus is used as the second terminal 20.

The memory 25 is a semiconductor memory for causing various kinds of control programs and the like executed by the apparatus (10 or 20), in addition to a digital image signal acquired by the image pickup portion 11 and various kinds of data transmitted/received via the apparatus communication portion 12, to be temporarily stored. As the memory 25, a temporary memory, for example, a rewritable nonvolatile semiconductor memory (for example, an EEPROM (electrically erasable programmable read-only memory)) or a volatile semiconductor memory used for temporary work (for example, an SDRAM (synchronous dynamic random access memory)) is also included.

Next, the main components of the file server 30 in the medical image management system 1 of the present embodiment will be described. Note that the file server 30 corresponds to, for example, a file server included in an existing hospital information system (HIS).

As shown in FIG. 6, the file server 30 is configured being provided mainly with the controlling portion 31, an image database 32, an order information database 33, a patient information database 34, a communication portion 35 and the like.

The controlling portion 31 is a control circuit configured to comprehensively control the file server 30. The controlling portion 31 is configured with a processor including hardware. The controlling portion 31 is configured mainly including a patient judging portion 31a, the order information creating portion 31b, a photographing condition information creating portion 31c and the like. Note that the controlling portion 31 is configured, for example, with a processor such as a CPU, an FPGA or the like.

The patient judging portion 31a is a circuit portion or program software configured to, based on patient-related information and the like included in request information or general order information transferred from the information terminal apparatus (10 or 20), perform association with patient information corresponding to the request information or the general order information.

The order information creating portion 31b is a circuit portion or program software configured to, when a doctor accesses the file server 30 to create order information, refer to various kinds of information of the image database 32, the order information database 33, the patient information database 34 and the like to support creation of the order information.

The order information creating portion 31b is a circuit portion or program software configured to create general order information according to request information in response to instruction information from a doctor (see the second use pattern in FIG. 3).

Here, for example, for general order information about image photographing, some format is specified for each affected part. Therefore, at the time of creating general order information, a doctor can create the general order information in a rough form by selecting a desired examination classification (for example, “image photographing examination”), a desired affected part (for example, “face” or “knee”) and the like.

Furthermore, in the medical image management system 1 of the present embodiment, for example, it is possible to refer to electronic medical record information, examination history information and the like about a target patient in the patient information database 34 and, if an order that is the same as or similar to a currently intended examination or the like (for example, “image photographing examination”) exists in a list of orders of examinations performed in the past, read the information to cause new general order information to be created.

As described above, the order information creating portion 31b is a component portion configured to, when a doctor creates order information (either medical order information or general order information), omit or improve efficiency of input work to support creation of the order information.

The photographing condition information creating portion 31c is a circuit portion or program software configured to, if order information created by the order information creating portion 31b based on an instruction from a doctor is, for example, an order involving image photographing, create photographing condition information suitable for the image photographing instruction.

The photographing condition information creating portion 31c refers to past order information at the time of similar “image photographing examinations” performed in the past, information about results of the examinations (such as acquired and recorded image data and accompanying information of the image data) and the like, based on patient information and the like referred to at the time of creating order information, and creates photographing condition setting information optimal for image photographing corresponding to current order information.

In a normal case, when a doctor creates order information, the doctor only gives a medical action instruction. In the medical image management system 1 of the present embodiment, however, the photographing condition information creating portion 31c creates photographing condition information for performing image photographing based on the order information configured only with the medical action instruction, which has been created by the doctor. The photographing condition information is communicated to the information terminal apparatus (10 or 20) for photographing (acquiring) image data in response to created general order information and used at the time of photographing.

An object information generating portion 31d is a circuit portion or program software for creating a medical object (a medical image data file), for example, as an object in a data format compliant with the DICOM standard.

More specifically, in response to medical order information or general order information issued from a doctor, the object information generating portion 31d extracts object information, which is various kinds of medical-related information to be given to an image data file corresponding to the order information (image data acquired according to the order information) as medical metadata, and generates medical metadata.

Further, the object information generating portion 31d associates, with an image data file (an Exif image data file) transferred to the image database 32 of the file server 30 after being acquired by the information terminal apparatus (10 or 20) according to medical order information or general order information, medical metadata generated from medical-related information (object information) extracted from the corresponding medical order information or general order information.

Then, the object information generating portion 31d associates an image data body with general metadata and medical metadata corresponding to the image data body to create one image data file as a medical object.

The image database 32 is a database portion in which image data files are accumulated. The plurality of image data files accumulated in the image database 32 include, in addition to various kinds of image data files included in each piece of specific patient information, for example, medical images or medical reference images obtained by photographing an affected part or symptom of a particular patient and an image including a face for identifying the patient, sample images and the like prepared in advance to be referred to at the time of acquiring an image of the particular affected part or particular symptom.

Here, the medical images or the medical reference images specifically include, for example, image data files in which a preoperative or postoperative course is recorded during a medical examination process, and image data files in which a state of an affected part during an operation is recorded. Forms of the image data files in this case include, in addition to an image data file recorded in a general image pickup apparatus (an Exif image data file), X-ray image data, ultrasound image data, endoscopic image data and the like. Among the above pieces of data, the X-ray image data, the ultrasound image data, the endoscopic image data and the like are image data acquired by various kinds of medical-related qualified workers using dedicated apparatuses installed in a medical base such as a hospital in response to doctors' orders (requests) and are medical images approved by the doctors. Such medical image data files basically have a data format compliant with the DICOM standard.

The data format is a configuration for clarifying what is a purpose of data, like the SOP class communication described before, and control for combination, rearrangement, rewriting and the like of data to cause the data to follow a way of communication in which data and information about how the data is to be handled are combined may be included. By performing conversion to such a data format, communication matching an existing system becomes possible. Though a terminal apparatus may perform such format conversion, a server may perform the format conversion instead.

Each of the plurality of image data files accumulated in the image database 32 except sample images is recorded in a form of being associated with corresponding medical-related information (such as electronic medical record information and patient information).

Furthermore, in each image data file, accompanying information (metadata) configured with general metadata including photographing date and time information and the like and medical metadata including a patient ID and various kinds of information based on electronic medical record information is recorded in association with an image data body.

The order information database 33 is a database portion in which information about order information issued by doctors is accumulated. As the information recorded to the order information database 33, there is sample information of order specification sheets set in advance, for example, for predetermined affected parts or predetermined symptoms. In each order specification sheet, for example, a doctor name (a requester name), a patient ID, used apparatus information and photographing condition information are written.

In general, in order for a doctor to perform an examination and the like for a particular patient, the doctor is required to issue order information appropriate for an affected part or a symptom for each target patient of the examination to each medical department at the time of requesting acquisition of image data.

In this case, there is an advantage that, since the order information database 33 is provided, the doctor does not have to create the order information to be newly created, from scratch.

The doctor can refer to an order specification sheet (a sample) corresponding to an affected part or a symptom of a currently targeted patient among the plurality of order specification sheets (samples) recorded in the order information database 33. Therefore, it becomes possible to create an order specification sheet optimal for each individual target patient according to the patient's symptom, affected part or the like.

The patient information database 34 is a database portion in which patient information, for example, patient IDs and electronic medical record information are accumulated.

The communication portion 35 is a circuit portion or program software configured to mutually perform transmission/reception of various kinds of data files with the plurality of information terminal apparatuses (10 and 20) via the network 40. The communication portion 35 is controlled by a communication controlling portion (not shown) included in the controlling portion 31 so that predetermined communication is executed.

Note that, in the above description, as a configuration for recording an image object (an image data file; an Exif file) acquired by a general image acquisition apparatus as a medical object (a medical image data file) in a data format compliant with the DICOM standard, a configuration of using the object information generating portion 31d included in the controlling portion 31 of the file server 30 is shown.

However, the configuration for recording an image object (an image data file; an Exif file) acquired using a general image acquisition apparatus as a medical object (a medical image data file) in a data format compliant with the DICOM standard is not limited to the above example.

For example, such a configuration is also possible that, at the time of image acquisition performed according to general order information issued by the doctor 110 in response to request information, in the information terminal apparatus used by the patient's family member or the like 120 (the second terminal 20), a medical image data file with which medical metadata is associated by the general order associating portion 21ea of the recording controlling portion 21e is created. In the case of the above configuration, the general order associating portion 21ea of the recording controlling portion 21e functions as an object information generating portion.

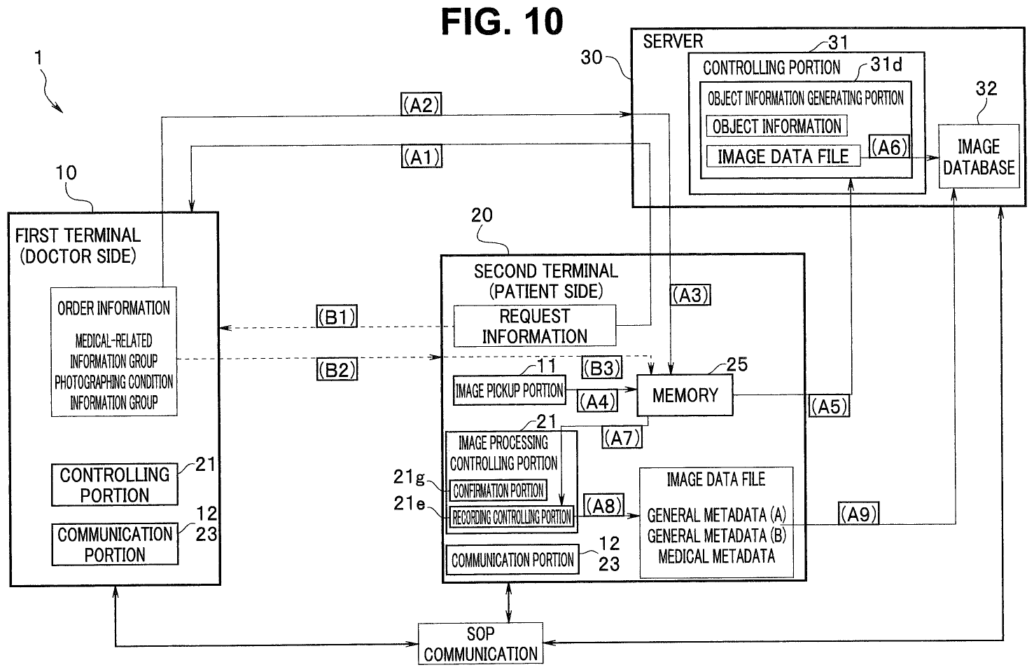

FIG. 10 is a conceptual diagram conceptually showing flows of various kinds of information in the medical image management system of the present embodiment.

Note that FIG. 10 simply shows a configuration of the medical image management system of the present embodiment to avoid complicatedness of the drawing. For example, the network 40 illustrated in FIG. 1 is not shown. Configurations of the first terminal 10 and the second terminal 20, which are information terminal apparatuses, are also simplified and shown.

FIG. 10 shows a configuration of the medical image management system for creating, sharing and managing a medical image data file; the first terminal can also be expressed as an information terminal configured to transmit medical-related particular-use information about a particular individual; and the second terminal can also be expressed as an image pickup apparatus configured to acquire an image data file. It is only required to provide a processor including hardware configured to associate medical service-related data obtained according to the above medical-related particular-use information and the above image data file to generate a medical image data file as a system, and the second terminal includes the configuration. Of course, such functions may be shared via communication within the system, and another apparatus may substitute. The configuration may be provided in a file server connected between the first information terminal apparatus and the second information terminal apparatus via a network.

In FIG. 10, two kinds of flows are roughly shown as flows of various kinds of information; that is, in FIG. 10, flows indicated by solid lines (symbol (Ax); x indicates a numeral) conceptually show information flows at the time of performing communication between the first terminal 10 and the second terminal 20 via the file server 30. In FIG. 10, flows indicated by broken lines (symbol (Bx); x indicates a numeral) conceptually show information flows at the time of performing direct communication between the first terminal 10 and the second terminal 20.

Note that, in description below, it is assumed that a person who uses the second terminal 20 is the patient's family member or the like 120 who is a non-medical worker. Therefore, it is described that order information issued by the doctor 110 is only general order information.

The second terminal 20 is a general image pickup apparatus which is privately owned by the patient's family member or the like 120 who is a non-medical worker. Usually, general photographing can be enjoyed with the second terminal 20 as a normal camera. Under a special situation, however, a worthwhile function can be activated.

However, for example, in a situation of the doctor performing visiting medical care or visiting nursing care, it may be a medical worker that uses the second terminal 20. In this case, order information issued by the doctor 110 in response to request information from the second terminal 20 is medical order information.

When communication between the first terminal 10 and the second terminal 20 is performed via the file server 30 in the medical image management system 1 of the present embodiment, the patient's family member or the like 120 transmits request information to the first terminal 10 from the second terminal 20 via the file server 30 first (symbol (A1) in FIG. 10).

When reading the requesting information, the doctor 110 operates the first terminal 10 to create general order information, and transmits the created general order information to the second terminal 20 from the first terminal 10 via the file server 30 (symbol (A2) in FIG. 10).

In response to the transmission, the second terminal 20 records the received general order information to the memory 25 (symbol (A3) in FIG. 10).

Next, the patient's family member or the like 120 uses the second terminal 20 to execute a photographing operation according to the received general order information. Consequently, the second terminal 20 generates a normal image data file compliant with the Exif standard based on an image signal acquired by the image pickup portion 11 and records the image data file to the memory 25 (symbol (A4) in FIG. 10).

Then, the general order information recorded to the memory 25 and the image data file (the Exif file) in a normal form acquired according to the general order information are transmitted to the object information generating portion 31d of the controlling portion 31 of the file server 30 (symbol (A5) in FIG. 10).

In response to the transmission, the object information generating portion 31d extracts predetermined medical-related information (object information) from the general order information to generate medical metadata, and associates the medical metadata with the corresponding image data file (the Exif image data file) to create one medical object (medical image data file).

The medical image data file created in this way is sent and recorded to the image database 32 (symbol (A6) in FIG. 10).

The following shows a case of a configuration in which a medical image data file is generated on the second terminal 20,

The process until general order information and an image data file (an Exif file) in a normal form acquired according to the general order information are recorded to the memory 25 is similar to the above case (symbols (A1) to (A4) in FIG. 10).

Next, the recording controlling portion 21e of the image processing controlling portion 21 generates medical metadata based on the general order information and the image data file (the Exif file) in the normal form acquired according to the general order information, which are recorded to the memory 25, and associates the medical metadata with the image data file to create a medical image data file in which an image data body, general metadata (A) and (B), and the medical metadata are integrated (symbol (A8) in FIG. 10).

The medical image data file created in this way is sent and recorded to the image database 32 (symbol (A9) in FIG. 10).

When communication between the first terminal 10 and the second terminal 20 is directly performed in the medical image management system 1 of the present embodiment, the patient's family member or the like 120 directly transmits request information to the first terminal 10 from the second terminal 20 first (symbol (B1) in FIG. 10).

When reading the requesting information, the doctor 110 operates the first terminal 10 to create general order information, and directly transmits the created general order information to the second terminal 20 from the first terminal 10 (symbol (B2) in FIG. 10).

In response to the transmission, the second terminal 20 records the received general order information to the memory 25 (symbol (B3) in FIG. 10).

Here, it is preferable that the information is correctly classified and recorded. For this purpose, each piece of information in the above order information can be divided and transmitted. If the order information is not transmitted being divided, a processor may be provided for performing classification into “medical-related particular-use information” and various kinds of information about an examination on “when”, “where”, “who”, “for whom” “which kind of apparatus”, “which kind of examination” and “under which photographing conditions”.

Such classification can be realized by an apparatus transmitting order information and an apparatus receiving the order information sharing information about what is indicated by which data, based on standards and the like, using a particular format and rules. Supplementary data for supplementarily describing, for each data, what kind of data the data is may be used so that each data group can be classified. Since it is possible to perform such classification, the processor can make it easy to refer to only necessary data and check deficiency and inconsistency of data.

It is not necessarily required to classify all such information. It is important to classify necessary information and make it possible to judge whether there is deficiency or inconsistency. In other words, by including a processor configured to judge whether a predetermined item actually corresponds to or is inconsistent with a situation or a target object at the time of photographing, and whether information to be obtained at the time of photographing has been correctly obtained, recorded and associated with an image, in the system, it is possible to judge whether photographed image data meets a requested quality and required specifications. If pieces of information are separated, it becomes possible to perform classification about for which service each of the pieces of information is important, by referring to a database or performing operation.

Next, the patient's family member or the like 120 uses the second terminal 20 to execute a photographing operation according to the received general order information. Consequently, the second terminal 20 generates a normal image data file compliant with the Exif standard based on an image signal acquired by the image pickup portion 11 and records the image data file to the memory 25 (similar to symbol (A4) in FIG. 10). Subsequent operation is similar to operation indicated by symbols (A5) to (A9) described above.

Note that, as shown in FIG. 10, at the time of performing communication via the network between the file server 30 and each information terminal apparatus (10 or 20), an SOP class (service-object pair class) is used (see FIG. 7).

Operation of the medical image management system 1 of the present embodiment configured as described above will be described below. Note that the operation described below basically assumes operation of the “second use pattern” or the “third use pattern” among the respective use patterns described above.

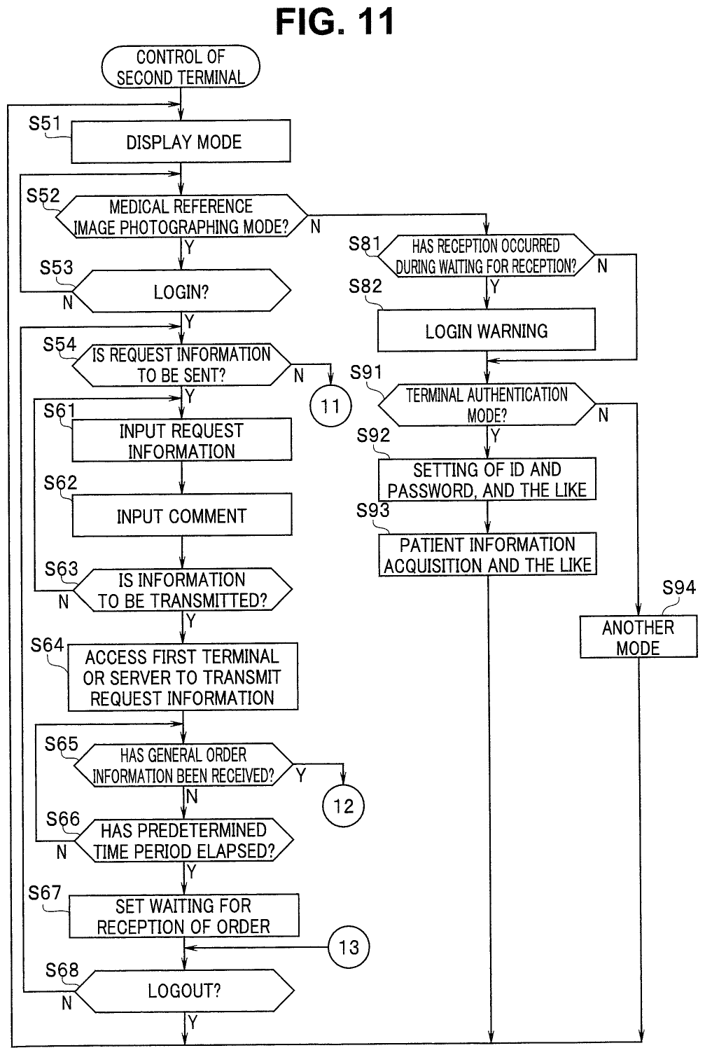

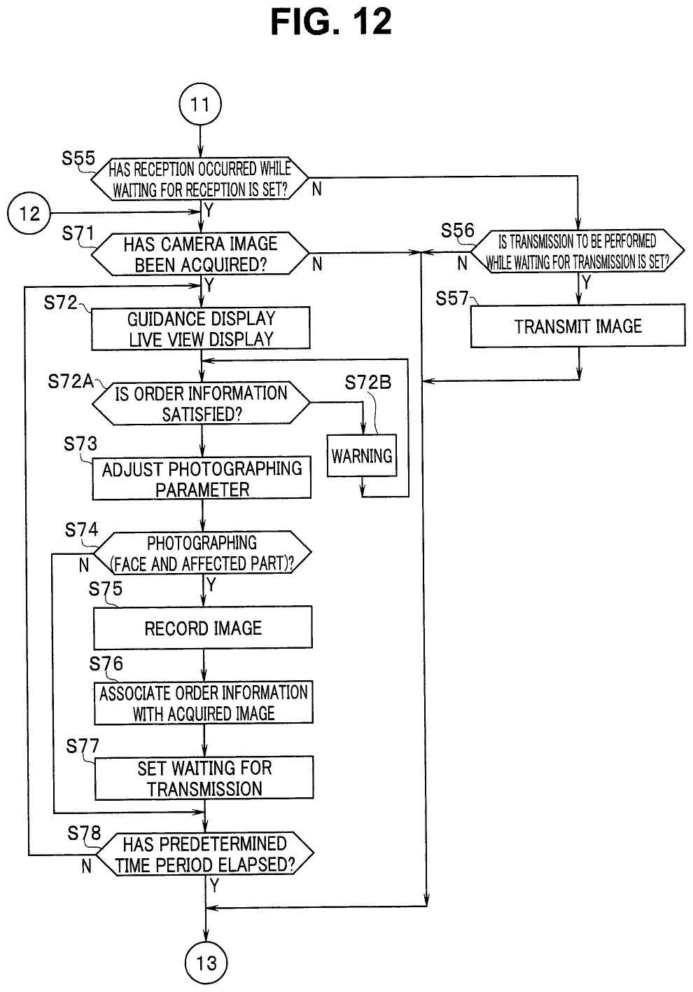

First, operation of the second terminal 20 among the information terminal apparatuses in the medical image management system 1 of the present embodiment will be described. FIGS. 11 and 12 are flowcharts showing the operation of the second terminal 20 among the information terminal apparatuses in the medical image management system of the present embodiment.

In the medical image management system 1 of the present embodiment, the second terminal 20 is assumed to be, for example, an information terminal apparatus used by a person other than a doctor, for example, a patient's family member as described above.

First, it is assumed that the second terminal 20 has been powered up and activated to be in a usable state.

In this state, first, the image processing controlling portion 21 of the second terminal 20 controls the display controlling portion 21d or the like to display an operation mode that is currently set, on a display screen of the display portion 15 at step S51 in FIG. 11. For example, as operation modes displayed here, that is, operation modes that can be executed by the second terminal 20, there are various kinds of operation modes executed by the “medical reference image acquisition application” described above, for example, the request information creation mode, the request information transmission mode, the general order information reception mode, the medical reference image photographing mode and the medical reference image transfer mode, in addition to a normal photographing mode, a reproduction mode, a various-kinds-of-settings mode and a mode for communication with other apparatuses and the like. Note that, in a normal case, an operation mode set by default immediately after the second terminal 20 is powered up is the photographing mode.

Then, at step S52, the image processing controlling portion 21 confirms the operation mode currently set and confirms whether the operation mode currently set is the medical reference image photographing mode or not. If the operation mode currently set is the medical reference image photographing mode, the image processing controlling portion 21 proceeds to processing of next step S53. If the operation mode currently set is an operation mode other than the medical reference image photographing mode, the image processing controlling portion 21 proceeds to processing of step S81.

At step S53, the image processing controlling portion 21 executes predetermined login processing. As the login processing, login processing for direct connection from the second terminal 20 to the first terminal 10 of a doctor or login processing for connecting to the file server 30 via the network 40 is assumed.

Note that an operation procedure for the login processing performed in the processing of step S53 described above is, for example, an operation of inputting information (for example, particular information such as a patient registration card number, a login ID, a login password and the like) issued from the doctor, a system on the hospital side or the like to a particular person (a patient or the patient's family member). More specifically, input means in this case is, for example, direct input by a touch operation on a numeric keypad display displayed on the display screen using the touch panel as an input operation member. An operation by image recognition input such as reading code information (a code in a form of a barcode or a QR code (registered trademark) including particular information such as a patient registration card number) issued from the doctor, the hospital or the like to the particular person (the patient or the patient's family member) using a code reading application or the like of the second terminal 20 is also possible.

The login processing here may be login processing for executing a series of operations in the medical reference image photographing mode using the second terminal 20. In other words, in order to execute a certain particular operation mode on the second terminal 20, predetermined login processing is to be performed at the time of executing the operation mode. By providing such login processing for an operation mode, it is possible to enable only a person for whom identity verification has been performed to cause the series of operations in the medical reference image photographing mode to be executed, using a terminal authenticated by the doctor or the hospital in advance.

When the login processing is completed here, the image processing controlling portion 21 proceeds to processing of next step S54. If the login processing is not completed here, the image processing controlling portion 21 returns to the processing of step S52 described above.

At step S54, the image processing controlling portion 21 confirms whether request information is to be sent or not. Here, if request information has not been sent yet and is to be sent, the image processing controlling portion 21 proceeds to processing of next step S61. If request information has already been sent, or if request information is not to be sent, the image processing controlling portion 21 proceeds to processing of step S55 in FIG. 12 (see circled reference numeral 11 in FIGS. 11 and 12).

If proceeding to step S61, assuming that request information is to be sent in the processing of step S54 described above, the image processing controlling portion 21 switches the operation mode to the request information creation mode and accepts processing for inputting basic information in the request information appropriately inputted from a user, at step S61. Then, at step S62, the image processing controlling portion 21 accepts processing for inputting comment information in the request information appropriately inputted from the user.

The request information inputted at steps S61 and S62 described above is, for example, information attached as comments in addition to the basic information identifying the patient, and it is assumed to provide information for consulting a current state of a disease and the like including a chief complaint, from the patient side to the doctor.

Whether or not the doctor who has received such request information sends general order information in response to the request information depends on the doctor's judgment. Therefore, there may be a possibility that, even if the request information is sent to the doctor from the patient side, a response from the doctor, for example, sending of general order information about an image, or the like is not performed. If the doctor considers that an image and the like of the patient are not necessary, such general order information is not sent.

Therefore, the request information created at steps S61 and S62 described above and sent to the doctor from the patient side as described later is considered much as consultation information to the doctor.

When the request information input processing ends in this way, the image processing controlling portion 21 proceeds to processing of next step S63.

At step S63, the image processing controlling portion 21 confirms whether processing for transmitting the created request information is to be executed or not. Here, if the request information transmission processing is to be executed, the image processing controlling portion 21 proceeds to processing of next step S64. If the request information transmission processing is not to be executed, for example, if additional input of the request information is to be performed, the image processing controlling portion 21 returns to the processing of step S61 and repeats the subsequent processing.

At step S64, the image processing controlling portion 21 switches the operation mode to the request information transmission mode. Then, the communication controlling portion 21c of the image processing controlling portion 21 controls the apparatus communication portion 12 or the network communication portion 23 to access the doctor's first terminal 10 or the file server 30 of a hospital system, and executes the request information transmission processing. After that, the image processing controlling portion 21 switches the operation mode to the general order information reception mode and enters a reception waiting state.

Next, at step S65, the image processing controlling portion 21 confirms whether general order information has been received or not. The general order information is information sent from the doctor's first terminal 10 or the file server 30 of the hospital system to the second terminal 20.

If the general order information has been received, the image processing controlling portion 21 proceeds to processing of step S71 in FIG. 12 (see circled reference numeral 12 in FIGS. 11 and 12). If the general order information has not been received, the image processing controlling portion 21 proceeds to processing of step S66.

At step S66, the image processing controlling portion 21 refers to the clock portion 22 to confirm whether a predetermined time period has elapsed or not. If the predetermined time period has elapsed, the image processing controlling portion 21 proceeds to processing of next step S67. If the predetermined time period has not elapsed, the image processing controlling portion 21 returns to the processing of step S65 described above and repeats the confirmation of reception.

If it is confirmed by the processing of step S66 described above that the predetermined time period has elapsed, and the image processing controlling portion 21 proceeds to the processing of step S67, then the image processing controlling portion 21 sets an order reception waiting mode at step S67. The order reception waiting mode is a reception waiting mode for receiving general order information as a response to the transmitted request information. In the second terminal 20, it is possible to, by setting the reception waiting mode, cause other desired work processing to be executed in parallel while being continuously in the reception waiting state. When setting of the order reception waiting mode is completed, the image processing controlling portion 21 proceeds to processing of next step S68.

At step S68, the image processing controlling portion 21 confirms whether logout processing is to be performed or not. If an instruction signal instructing logout processing is confirmed, the image processing controlling portion 21 proceeds to the processing of step S51 described above after executing predetermined logout processing. If the instruction signal instructing logout processing is not confirmed, the image processing controlling portion 21 returns to the processing of step S54 described above and repeats the subsequent processing.

On the other hand, if it is confirmed that the operation mode currently set is an operation mode other than the medical reference image photographing mode in the processing of step S52 described above, and the image processing controlling portion 21 proceeds to the processing of step S81, then the image processing controlling portion 21 confirms at step S81 whether reception of general order information has been performed or not. In a case where reception of general order information is confirmed here, the image processing controlling portion 21 proceeds to processing of next step S82. In a case where reception of general order information is not confirmed, the image processing controlling portion 21 proceeds to processing of step S91. Note that the case where reception of general order information is confirmed is the case where the order reception waiting mode is set by the processing of step S67 described above.

At step S82, the display controlling portion 21d of the image processing controlling portion 21 controls the display portion 15 to display a predetermined login warning on the display screen of the display portion 15. The login warning is display that prompts the user to perform a login operation for logging in the first terminal 10 or the file server 30 to acquire the received general order information.

Note that the above description of step S81 is made using a sentence that general order information has been received. However, actually, a substantial part of the information has not been received in the processing of step S81. It is meant that, at the point of time of step S81, for example, a mere notification to the effect that general order information has been sent from the doctor or the file server 30 to the second terminal 20 has been received. Therefore, in order to receive the substance of the general order information, the login processing is performed at step S82 described above.

After appropriately performing a login operation, the user of the second terminal 20 performs a predetermined operation to receive the general order information. Note that, as described above, the order reception waiting mode is executed in parallel with other operation processing. Therefore, after the general order information reception processing ends, the user can return to the other operation processing.

When it is confirmed that the operation mode currently set is a mode other than the medical reference image photographing mode in the processing of step S52 described above, the processing branches to the processing of step S81, and, therefore, another operation following the processing of step S82 described above is executed by transition to processing of step S91.

At step S91, the image processing controlling portion 21 confirms whether the operation mode currently set is a terminal authentication mode or not. Here, the terminal authentication mode is an operation mode for performing a registration procedure and the like for enabling the second terminal 20 to be used in the medical image management system 1.

Note that the registration procedure in the terminal authentication mode is an operation required to be performed once first at the time of using an information terminal apparatus (the second terminal 20) in the medical image management system 1. At the time of using a registered information terminal apparatus second time and subsequently, the information terminal apparatus can be used in the medical image management system 1 by using an ID and a password set and acquired by the first registration procedure.

Here, if it is confirmed that the mode is set to the terminal authentication mode, the image processing controlling portion 21 proceeds to processing of next step S92. If the mode is not the terminal authentication mode, the image processing controlling portion 21 proceeds to processing of step S94.

Then, at step S94, the image processing controlling portion 21 executes processing in another operation mode. Note that, as the other mode, a telephone communication mode, a mail transmission/reception mode or the like is conceivable in the case of an image-pickup-function-equipped communication apparatus (a device such as a smartphone). In the case of a communication-function-equipped image pickup apparatus (a device such as a digital camera), a normal photographing mode, a reproduction mode, a menu mode (a setting mode) or the like is conceivable.

However, since the other operation modes are not directly related to the present invention, details of the operation modes are not described or shown. Therefore, as for the subsequent processing, it is assumed that the image processing controlling portion 21 returns to the processing of step S51 described above.

As described above, if it is confirmed that the terminal authentication mode is set, and the image processing controlling portion 21 proceeds to the processing of step S92, then the image processing controlling portion 21 executes processing related to a registration procedure required for terminal authentication, such as ID setting processing and password setting processing, at step S92. After that, the image processing controlling portion 21 proceeds to processing of step S93.

At step S93, the operation judging portion 21b of the image processing controlling portion 21 executes processing for acquiring patient information and the like about the target patient from the file server 30, and the like in response to an operation instruction signal inputted from the operation portion 13 or the touch panel 14. When the necessary processing ends, the image processing controlling portion 21 waits for an end instruction signal, returns to the processing of step S51 described above and repeats the subsequent processing.

On the other hand, if request information is not to be sent at step S54 described above, and the image processing controlling portion 21 proceeds to step S55 in FIG. 12, then the image processing controlling portion 21 confirms at step S55 whether reception of general order information has been performed or not. If reception of general order information is confirmed here, the image processing controlling portion 21 proceeds to processing of next step S71. In this case, since the login state is maintained, the substance of the general order information has been received.

If reception of general order information is not confirmed, the image processing controlling portion 21 proceeds to processing of step S56. Here, the case where the image processing controlling portion 21 proceeds to the processing of step S56 occurs in a situation shown below. The situation is a situation in which it has been confirmed by the processing of step S52 described above that the mode is the medical reference image photographing mode, login has been completed by the processing of step S53 described above, it has been confirmed by the processing of step S54 described above that request information is not to be sent, and it has been confirmed by the processing of step S55 described above that general order information has not been received. As such a situation, for example, a case is conceivable where the series of operations in the medical reference image photographing mode (transmission of request information, reception of general order information and photographing in the medical reference image photographing mode) has been already performed, and an image data file as a desired medical reference image has been already acquired. In such a case, the image processing controlling portion 21 proceeds to the processing of step S56, and operation processing in the medical reference image transfer mode is executed (which is to be described later).

Note that the case where reception of general order information is confirmed is the case where the order reception waiting mode has been set by the processing of step S67 described above, similarly to the processing of step S81 described above.

When reception of general order information is confirmed, the image processing controlling portion 21 of the second terminal 20 switches the operation mode to the medical reference image photographing mode. The user (the patient's family member) of the second terminal 20 executes a predetermined photographing operation according to the received general order information using the second terminal 20.

At step S71, the image processing controlling portion 21 confirms whether or not predetermined photographing processing has been executed, and an image data file has been acquired. If acquisition of an image data file is confirmed, the image processing controlling portion 21 proceeds to processing of step S72. If acquisition of an image data file is not confirmed (in a case where photographing has not been performed yet), the image processing controlling portion 21 returns to the processing of step S68 in FIG. 11 (see circled reference numeral 13 in FIGS. 11 and 12).

When acquisition of an image data file is confirmed, the image processing controlling portion 21 displays guidance information (for example, a photographing procedure or display of information) and comment information included in the received general order information and performs live view display for sequentially displaying an image data file acquired by the image pickup portion 11 at step S72. In this case, the general order information and the live view display may be displayed in chronological order, may be overlappedly displayed or may be switchedly displayed by appropriately operating an operation member.

Then, at step S72A, the image processing controlling portion 21 (the confirmation portion 21g) confirms whether photographing according to the general order information is going to be performed or not, that is, whether various kinds of instructions included in the general order information are satisfied or not, based on the various kinds of information included in the general order information and various kinds of information included in the image data file of the live view display image (detection of the confirmation sensor 17). Here, if it is confirmed that the various kinds of instructions included in the general order information are satisfied, the image processing controlling portion 21 proceeds to processing of next step S73. If it is confirmed that the various kinds of instructions included in the general order information are not satisfied, the image processing controlling portion 21 proceeds to processing of step S72B.

At step S72B, the image processing controlling portion 21 controls the display portion 15 via the display controlling portion 21d to display a predetermined warning to the effect that the instructions in the order information is not satisfied. After that, the image processing controlling portion 21 returns to the processing of step S72.

Here, the user (the patient's family member) of the second terminal 20 corrects an operation so that photographing according to the order information is performed, in response to the warning display. If a correct operation is performed, and it is confirmed by the processing of step S72A that the instructions in the order information are satisfied, then the warning display goes out. The image processing controlling portion 21 thereby proceeds to processing of next step S73.

Then, at step S73, the image processing controlling portion 21 executes processing for adjusting photographing parameters and the like in the second terminal 20 in consideration of information such as photographing conditions included in the received general order information.

Next, at step S74, the image processing controlling portion 21 confirms whether photographing according to the general order information (for example, a face image and an affected part image of the patient) has been performed or not.

Here, for example, the face image of the patient is acquired together with the desired affected part image. This is for the purpose of confirming whether or not the desired affected part image photographed at the same time is an image of the patient himself/herself. The user is notified that such an identity verification image is simultaneously acquired at the time of acquiring a medical reference image, by the processing of step S72 described above or the like, for example, based on various kinds of instructions included in the guidance information or the like included in the general order information. Since the user performs photographing according to the guidance information, the user does not forget to acquire such an identity verification image as described above together with a desired affected part image.

When confirming that photographing according to the general order information has been performed, by the processing of step S74 described above, the image processing controlling portion 21 proceeds to processing of next step S75. When confirming that photographing according to the general order information has not been performed, the image processing controlling portion 21 proceeds to processing of step S78.

At step S75, the recording controlling portion 21e of the image processing controlling portion 21 executes processing for recording the acquired image data file.

Then, at step S76, the recording controlling portion 21e of the image processing controlling portion 21 executes association processing for associating the various kinds of information included in the general order information with the acquired image data file.

The association processing performed here is specifically processing for generating one integrated image data file in which medical metadata is associated with an image data file acquired by the image pickup portion 11 (an Exif image data file in which an image data body and general metadata are integrated), which is a medical object (a medical image data file). The medical image data file generated in this way is recorded as an image data file in a form of various kinds of information being arranged and collected, for example, in a data format compliant with the DICOM.

Next, at step S77, the image processing controlling portion 21 sets an image transmission waiting mode for transmitting an image data file of a medical reference image. The image transmission waiting mode is a transmission waiting mode for transmitting an image data file photographed according to received general order information (a medical reference image). In the second terminal 20, it is possible to, by setting the transmission waiting mode, cause other desired work processing to be executed in parallel while being continuously in a transmission waiting state. When setting of the image transmission waiting mode is completed, the image processing controlling portion 21 proceeds to processing of next step S78.

Note that, as a situation in which the image transmission waiting mode is set, a case is conceivable where the second terminal 20 is in an environment in which communication with the first terminal 10 or the file server 30 is impossible. In such a case, the image transmission waiting mode is set, and, at the time of having moved to a place in a good communication environment, communication can be secured, and transmission can be performed.

At step S78, the image processing controlling portion 21 refers to the clock portion 22 to confirm whether a predetermined time period has elapsed or not. If the predetermined time period has elapsed, the image processing controlling portion 21 proceeds to processing of step S68 in FIG. 11 (see circled reference numeral 13 in FIGS. 11 and 12). If the predetermined time period has not elapsed, the image processing controlling portion 21 returns to the processing of step S72 described above and repeats the subsequent processing.

If reception of general order information is not performed in processing at step S55 described above, and the image processing controlling portion 21 proceeds to the processing of step S56, then the image processing controlling portion 21 confirms at step S56 whether processing for transmitting the acquired image data file of the medical reference image has been executed or not. Note that it is about whether execution of the transmission processing is possible or not that is confirmed here. If execution of the transmission processing is possible here, the image processing controlling portion 21 proceeds to processing of next step S57.

Then, at step S57, the image processing controlling portion 21 executes predetermined image transmission processing after switching the operation mode to the medical reference image transfer mode. After that, the image processing controlling portion 21 returns to the processing of step S68 in FIG. 11 (see circled reference numeral 13 in FIGS. 11 and 12).

On the other hand, if it is confirmed that execution of the transmission processing is impossible in the processing of step S56 described above, the image processing controlling portion 21 also returns to the processing of step S68 in FIG. 11 (see circled reference numeral 13 in FIGS. 11 and 12).

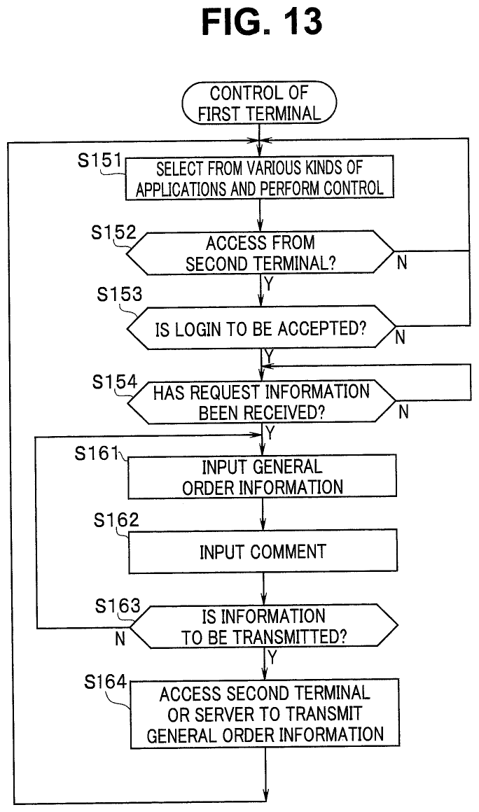

Next, operation of the first terminal 10 among the information terminal apparatuses in the medical image management system 1 of the present embodiment will be described. FIG. 13 is a flowchart showing the operation of the first terminal 10 among the information terminal apparatuses in the medical image management system of the present embodiment.

Description will be made on an assumption that, in the medical image management system 1 of the present embodiment, the first terminal 10 is an information terminal apparatus (more specifically, for example, a tablet PC) which is, for example, used by a doctor as described above.

First, the image processing controlling portion 21 of the first terminal 10 controls the display controlling portion 21d or the like to display a plurality of icons indicating various kinds of executable application software on the display screen of the display portion 15 at step S151 in FIG. 13 so that it is possible to respond to work using various applications.

For example, it is possible to perform telephone contact, e-mailing or chat contact with clerical workers, medical workers and involved persons inside and outside a medical facility. By the terminal, it is possible to select not only schedule management or reading of patient information but also search for various kinds of information from among icons. It is possible to easily create a work record and the like making full use of such applications. Since a dictation application and the like may be used for creation of a document nowadays, the terminal is compatible with such a technique.

It is preferable that the terminal enables not only the above state but also access from an external apparatus. Consequently, the doctor can respond to an emergency patient by the terminal or can respond to inquiries from other medical workers. Here, description is made by an expression that one of the applications executing the processing of step S151 has a waiting function described below though the function is expressed in a form of following step S151. However, the present invention is not limited to such a configuration. The object of the present invention can be achieved by such a design that applications work in parallel.

At step S152, the image processing controlling portion 21 confirms whether or not there is access, for example, from the second terminal 20. Though the applications at step S151 include various functions other than the access confirmation, description is simplified here. Since a characteristic part of the present invention can be described by S152 and subsequent steps, description will be made below in a form of especially mentioning the characteristic part of the present invention.

When access from the second terminal 20 is confirmed at step S152, the image processing controlling portion 21 proceeds to processing of next step S153. If access from the second terminal 20 is not confirmed, the image processing controlling portion 21 returns to the processing of step S151.

Note that when being in the waiting state, the first terminal 10 can accept other various kinds of input instructions and the like even if access from the second terminal 20 is not confirmed. However, operations corresponding to such other input instructions are not directly related to the present invention. Therefore, it is assumed that, if access from the second terminal 20 is not confirmed by the processing of step S152, the image processing controlling portion 21 returns to the processing of step S151 for convenience, without describing the operations corresponding to the other input instructions.

Then, at step S153, the image processing controlling portion 21 confirms whether login procedure processing (the login processing at step S53 in FIG. 11) from the second terminal 20 is authorized or not, that is, whether the login is to be accepted or not. If the login is to be accepted, the image processing controlling portion 21 proceeds to processing of next step S154. If the login is not to be accepted, the image processing controlling portion 21 returns to the processing of step S151. Here, the case where the login is not to be accepted is a case where information included in the login procedure, for example, a login ID or a password does not match, or the like.

When the login is accepted, and the image processing controlling portion 21 proceeds to processing of step S154, the image processing controlling portion 21 waits for reception of information after the login. Since there may be abrupt communication disconnection or arrival of irregular information, it may be designed to wait only for a predetermined time period, or some warning may be issued. However, a simplified expression is adopted here. The image processing controlling portion 21 switches to a request information reception mode for receiving request information. Then, the image processing controlling portion 21 receives transmitted request information and confirms whether the request information has been received or not (processing corresponding to the request information transmission by the processing of step S54 in FIG. 11). Here, the image processing controlling portion 21 repeats the reception confirmation processing until request information is received. If request information is received, the image processing controlling portion 21 proceeds to processing of next step S161. Note that when a predetermined time period elapses without receiving request information, the image processing controlling portion 21 may exit the loop and return to the processing of step S151, though this is not shown in the flowchart of FIG. 13.

Next, at step S161, the image processing controlling portion 21 switches to the general order information creation mode and waits for a user's input instruction. A user appropriately inputs corresponding general order information according to the request information received by the processing of step S154 described above.

Then, at step S162, the image processing controlling portion 21 accepts comment information input processing for inputting arbitrary comments in the general order information appropriately inputted by the user.

The general order information inputted at steps S161 and S162 described above is, for example, in addition to basic information for identifying a patient, information given as comments, which is information issued by the doctor in response to request information from a patient's family member, and various kinds of information including image photographing instruction information and the like.

When the general order information input processing ends in this way, the image processing controlling portion 21 proceeds to processing of next step S163.

At step S163, the image processing controlling portion 21 confirms whether or not to execute processing for transmitting the created general order information. Here, if the general order information transmission processing is to be executed, the image processing controlling portion 21 proceeds to processing of next step S164. If the general order information transmission processing is not to be executed, for example, if additional input to the created general order information is to be performed, the image processing controlling portion 21 returns to the processing of step S161 and repeats the subsequent processing.

At step S164, the image processing controlling portion 21 switches the operation mode to the general order information transmission mode. Then, the communication controlling portion 21c of the image processing controlling portion 21 controls the apparatus communication portion 12 or the network communication portion 23 to access the second terminal 20 of the patient's family member or the file server 30 of the hospital system and execute processing for transmitting the created general order information. After that, the image processing controlling portion 21 switches the operation mode to a normal waiting state.

Note that the first terminal 10 can operate in various operation modes other than the operation modes described above. However, since the other operation modes of the first terminal 10 are not directly related to the present invention, description of the operation modes will be omitted.

In the flowchart shown in FIG. 13 also, only such parts that are necessary to describe the operation of the present invention are shown, and parts that are not directly related to the present invention are not shown or described. As for operations of the parts that are not described, it is assumed that operations similar to operations of a conventional general information terminal apparatus (a tablet PC is assumed in the present embodiment) can be realized.

Furthermore, in the first terminal 10, it is also similar to a conventional information terminal apparatus of the same type that it is possible to realize various operations depending on installed application software.

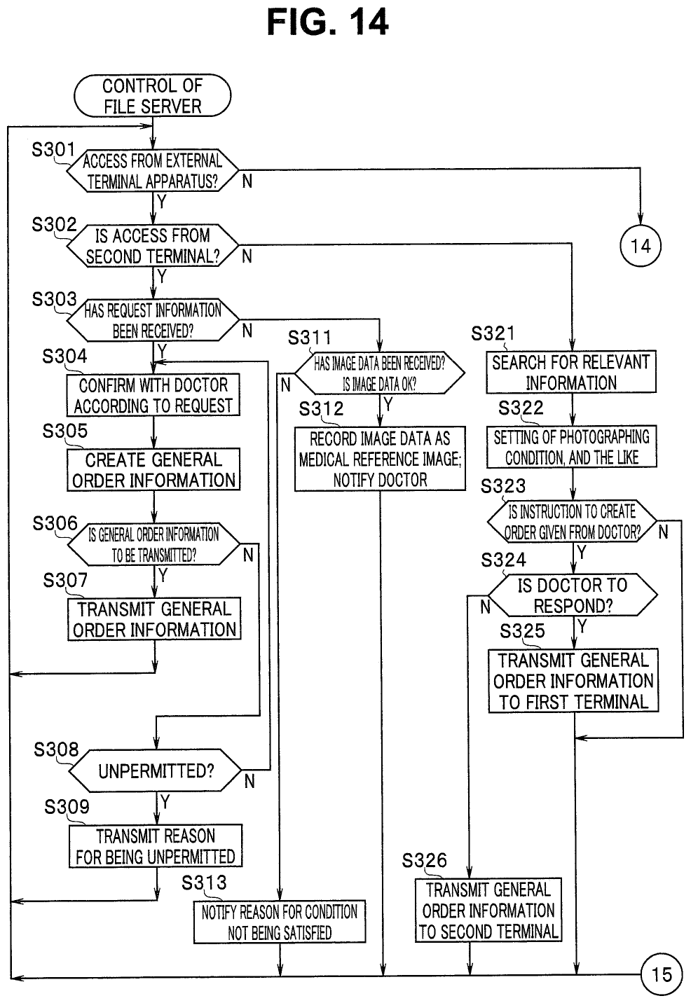

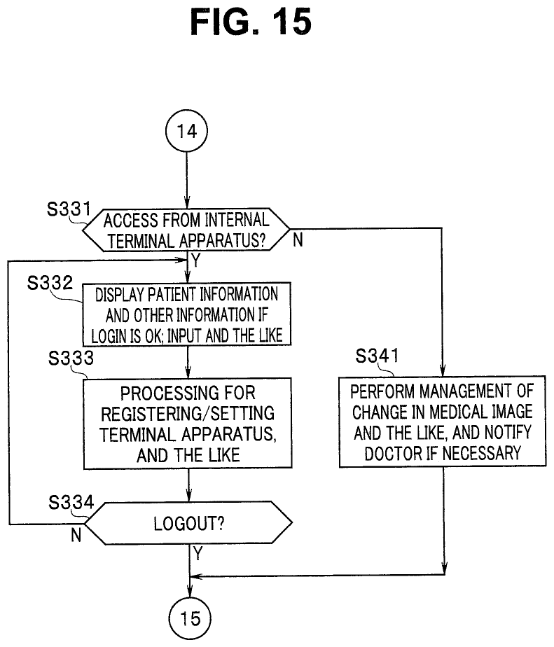

Next, operation of the file server 30 in the medical image management system 1 of the present embodiment will be described. FIGS. 14 and 15 are flowcharts showing the operation of the file server in the medical image management system of the present embodiment.

First, it is assumed that the file server 30 is in an activated state and in a waiting state of waiting for access from another apparatus such as an external information terminal apparatus or an internal terminal apparatus.

Here, the external information terminal apparatus refers to the first terminal 10, the second terminal 20 or the like. Note that, in FIG. 14, the first terminal 10, the second terminal 20 or the like is simply expressed as an external terminal apparatus. The internal terminal apparatus refers to a PC or the like installed in a reception department or a medical examination room in a medical facility (not shown). Therefore, the file server 30 allows access not only from the external information terminal apparatuses (10, 20 and the like) but also from the internal terminal apparatuses.

When the file server 30 is in this state, the communication portion 35 of the controlling portion 31 confirms whether there is access from an external information terminal apparatus (10, 20 or the like) at step S301 in FIG. 14. If access from an external information terminal apparatus (10, 20 or the like) is confirmed, the communication portion 35 proceeds to processing of next step S302. If access from an apparatus other than the external information terminal apparatuses (10, 20 and the like) is confirmed, the communication portion 35 proceeds to processing of step S331 in FIG. 15 (see circled reference numeral 14 in FIGS. 14 and 15).

At step S302, the communication portion 35 of the controlling portion 31 confirms whether the access is from the second terminal 20 or not. If access from the second terminal 20 is confirmed, the communication portion 35 proceeds to processing of next step S303 after executing predetermined login processing. If access from an apparatus other than the second terminal 20 (that is, the first terminal 10) is confirmed, the communication portion 35 proceeds to processing of step S321 after executing predetermined login processing.

At step S303, the controlling portion 31 confirms whether request information from the second terminal 20 has been received or not. If it is confirmed that request information has been received, the controlling portion 31 proceeds to processing of step S304. If request information has not been received, the controlling portion 31 proceeds to processing of step S311.

At step S304, the controlling portion 31 confirms with a corresponding doctor according to the received request information. The processing performed here corresponds to, for example, processing for transferring the received request information to the first terminal 10 of the corresponding doctor. The doctor who receives the request information performs a predetermined operation using his/her own first terminal 10 though it is not shown.

Next, at step S305, the controlling portion 31 creates general order information based on instruction information received from the doctor in the processing of step S304 described above.

Then, at step S306, the controlling portion 31 confirms whether a transmission instruction signal is issued or not, the transmission instruction signal indicating whether or not the general order information created in the processing of step S305 described above is to be transmitted to the second terminal 20, which is a sender of the received request information. The transmission instruction signal is an instruction signal issued from the doctor.

If the general order information transmission instruction signal is confirmed here, the controlling portion 31 proceeds to processing of next step S307. If the general order information transmission instruction signal is not confirmed, the communication portion 35 proceeds to processing of step S308.

At step S307, the controlling portion 31 executes transmission processing for transmitting the general order information created by the processing of step S305 described above to the second terminal 20, which is the sender of the received request information. After that, the controlling portion 31 returns to the processing of step S301 and repeats the subsequent processing. The file server 30 enters the state of waiting for access from an external apparatus.

On the other hand, if the general order information transmission instruction signal is not confirmed in the processing of step S306 described above, and the controlling portion 31 proceeds to the processing of step S308, then the controlling portion 31 confirms whether transmission can be permitted or not. Whether transmission can be permitted or not can be checked, for example, by whether or not a checkmark is put on the permission display 10b (see FIGS. 2 and 3) included in the general order information created in the processing of step S305 described above.

If information to the effect that transmission is permitted is confirmed here, the controlling portion 31 returns to the processing of step S304 described above and repeats the subsequent processing. If the information to the effect that transmission is permitted is not confirmed, that is, if there is information to the effect that transmission is not permitted, the controlling portion 31 proceeds to processing of step S309.

At step S309, the controlling portion 31 transmits information including a reason why transmission is not permitted to the second terminal 20, which is the sender of the received request information. After that, the controlling portion 31 returns to the processing of step S301 and repeats the subsequent processing. In other words, the file server 30 enters the state of waiting for access from an external apparatus.

On the other hand, if access from an apparatus other than the external information terminal apparatuses (10, 20 and the like) is confirmed in the processing of step S301 described above, and the controlling portion 31 proceeds to the processing of step S331 in FIG. 15, then the controlling portion 31 confirms whether or not there is access from an internal terminal apparatus (not shown; for example, an internal terminal apparatus (not shown) such as a PC installed in the reception department, or a medical examination room in the medical facility) at step S331. If access from an internal terminal apparatus is confirmed, the controlling portion 31 proceeds to processing of next step S332. If access from an internal terminal apparatus is not confirmed, the controlling portion 31 proceeds to processing of step S341.

At step S332, the controlling portion 31 executes predetermined login procedure processing. In response to the execution, the internal terminal apparatus (not shown) performs predetermined login procedure processing. When login to the file server 30 is completed (in a case where login is OK), the controlling portion 31 transfers predetermined information to the internal terminal apparatus in response to a request from the internal terminal apparatus. In response to the transfer, the internal terminal apparatus displays the predetermined information on a display portion (not shown). The controlling portion 31 performs information recording processing in response to input of information from the internal terminal apparatus (for example, input for updating an electronic medical record). After that, the controlling portion 31 proceeds to processing of step S333.

Note that if the internal terminal apparatus cannot log in the file server 30, the file server 30 cancels the subsequent processing, returns to the processing of step S301 described above and enters the waiting state, though it is not shown.

At step S333, in response to an operation instruction from the internal terminal apparatus, the controlling portion 31 executes processing for registering/setting an information terminal apparatus (10, 20 or the like), and the like.

Then, at step S334, the controlling portion 31 confirms a logout instruction from the access source apparatus. If a logout instruction is confirmed here, the controlling portion 31 returns to the processing of step S301 of FIG. 14 after executing predetermined logout processing, and repeats the subsequent processing.

On the other hand, if access from an internal terminal apparatus is not confirmed by the processing of step S331 described above, the controlling portion 31 proceeds to processing of step S341. At this point of time, it is already known that access from an external terminal apparatus has not been confirmed, in the processing of step S301 described above (FIG. 14).

Therefore, in this case, the file server 30 is in the waiting state of waiting for access from another apparatus.

At the same time, at step S341, the controlling portion 31 of the file server 30 executes processing such as processing for performing various kinds of analyses, for example, based on a plurality of image data files accumulated in the image database 32 and a plurality of pieces of patient information recorded in the patient information database 34 and notifying a doctor's first terminal of results of the analyses as necessary.

More specifically, for example, analysis about progress of a disease condition based on change over time is performed in terms of a plurality of image data files associated with particular patient information. Such analysis processing can be realized, for example, by utilizing AI and the like.

On the other hand, if access from an apparatus other than the second terminal 20 (that is, the first terminal 10) is confirmed in the processing of step S302 described above, and the controlling portion 31 proceeds to the processing of step S321, then the controlling portion 31 executes relevant information search processing at step S321. The relevant information search processing is processing for responding to an instruction given by a doctor to search for necessary information, for example, patient information such as an electronic medical record about a patient that the doctor is in charge of (in this case, a patient or the like who is a sender of request information) using the first terminal 10.

Then, at step S322, the controlling portion 31 makes photographic condition settings and the like for photographing of an image according to the request information. As for the processing performed here, there are a case where the doctor may perform operation input using the first terminal 10 to specify various kinds of settings and a case where the controlling portion 31 (the order information creating portion 31b) of the file server 30 may automatically make the various kinds of settings.

Next, at step S323, the controlling portion 31 confirms whether or not an instruction to create general order information is given from the doctor's first terminal 10. If the general order information creation instruction from the doctor is confirmed here, the controlling portion 31 proceeds to processing of next step S324. If the general order information creation instruction from the doctor is not confirmed, the controlling portion 31 judges that the doctor does not issue general order information according to the request information, returns to the processing of step S301 described above and enters the waiting state.

On the other hand, if the general order information creation instruction from the doctor is confirmed in the processing of step S323 described above, and the controlling portion 31 proceeds to the processing of step S324, then the controlling portion 31 confirms whether the doctor responds or not, at step S324. In this case, the response by the doctor refers to, for example, a case where, at the time of creating general order information corresponding to the request information using the first terminal 10, the doctor performs input for each item himself/herself.

If an instruction signal to the effect that the doctor responds is confirmed, the controlling portion 31 proceeds to processing of next step S325, and the controlling portion 31 transmits the created general order information to the first terminal 10 at step S325. After that, the controlling portion 31 returns to the processing of step S301 described above and enters the waiting state.

On the other hand, if the instruction signal to the effect that the doctor responds is not confirmed, the controlling portion 31 proceeds to processing of step S326, and the controlling portion 31 transmits the created general order information to the second terminal 20 at step S326. After that, the controlling portion 31 returns to the processing of step S301 described above and enters the waiting state.

If it is confirmed that request information has not been received in the processing of step S303 described above, and the controlling portion 31 proceeds to processing of step S311, then the controlling portion 31 confirms whether an image data file from the second terminal 20 has been received or not, at step S311. If reception of an image data file is confirmed, the controlling portion 31 confirms whether the image data file satisfies predetermined conditions or not. The image data file sent from the second terminal 20 is an image data file obtained by performing photographing according to the general order information. The predetermined conditions for the image data file are conditions specifying that photographing should be performed according to the general order information, for example, conditions that a predetermined image format should be adopted and that predetermined accompanying information should be attached.

If it is confirmed that an image data file has been received, and it is confirmed that the image data file satisfies the predetermined conditions, the controlling portion 31 proceeds to processing of next step S312. If reception of an image data file is not confirmed or if, though reception of an image data file is confirmed, it is confirmed that the image data file does not satisfy the predetermined conditions, the controlling portion 31 proceeds to processing of step S313.

At step S312, the controlling portion 31 records the image data file to a predetermined area (for example, the image database 32) as a medical reference image and as patient information. At this time, the image data file to be recorded is associated with corresponding particular patient information (an electronic medical record and the like). Then, a notification to the effect that a medical reference image corresponding to the general order information has been recorded is transmitted to the doctor's first terminal 10. After that, the controlling portion 31 returns to the processing of step S301 described above and enters the waiting state.

At step S313, the controlling portion 31 notifies the second terminal 20 that has transmitted the image data file of a reason for the conditions not being satisfied, and the like. Here, the reason for the conditions not being satisfied is a reason why the received image data file cannot be adopted as a medical reference image as a result of the confirmations in the processing of step S311 described above, that is, predetermined conditions that are not satisfied. After that, the controlling portion 31 returns to the processing of step S301 and enters the waiting state.

As described above, according to the one embodiment, the medical image management system 1 for sharing patient information to support medical services is configured being provided with a first information terminal apparatus (the first terminal 10) having a communication function and an image pickup function and is configured to transmit particular-use information (general order information) according to received request information, and a second information terminal apparatus (the second terminal 20) having at least a communication function and is configured to transmit inputted request information, and the medical image management system 1 being configured so that the second information terminal apparatus records, at the time of recording an acquired image data file, the particular-use information received from the first information terminal apparatus in association with the image data file.

In the medical image management system 1, when a non-medical worker such as a patient's family member transmits predetermined request information to the first information terminal apparatus (the first terminal 10) that a doctor has or the file server 30 using the second information terminal apparatus (the second terminal 20), the doctor performs, in response to the transmission, a predetermined operation according to the received request information using the first information terminal apparatus (the first terminal 10). Consequently, particular-use information (general order information) is created in the first information terminal apparatus (the first terminal 10) or the file server 30.

The particular-use information (general order information) created in this way is transmitted to the second information terminal apparatus (the second terminal 20). In response to the particular-use information, the non-medical worker such as a patient's family member performs photographing of an image using the second information terminal apparatus (the second terminal 20) to acquire an image data file of a target patient. Here, the second information terminal apparatus (the second terminal 20) records the acquired image data file in association with the particular-use information (general order information) received from the first information terminal apparatus (the first terminal 10) or the file server 30.

In other words, the second terminal 20 associates medical metadata based on medical-related information included in general order information with an acquired image data file (an Exif image data file in which an image data body and general metadata are integrated) to generate one integrated medical object (a medical image data file). Consequently, the acquired image data file is in a state capable of being approved as a medical reference image and becomes an image data file, for example, in a data format compliant with the DICOM standard.

Note that, as for the processing for associating an acquired image data file (an Exif file) and medical metadata, for example, a configuration in which the processing is performed in the file server 30 may be adopted in addition to the configuration in which the processing is performed in the second terminal 20, as described above.

Thus, if information including an image photographing instruction and the like is included in general order information (a group of pieces of information equivalent to order information including a plurality of medical action instructions) as particular-use information that is issued by a doctor according to request information from a patient's family member, and photographing of an image is performed using the second terminal 20 which has received the general order information, then various kinds of information included in the general order information are associated with an acquired image data file (an Exif file) as medical metadata, and the image data file is recorded as a medical image data file in a data format compliant with DICOM.

The image data file created in this way is shared as a reliable medical reference image by being transferred to the file server 30 or the like.

Therefore, thereby, if a patient's family member or the like at a remote location transmits predetermined request information to a doctor and receives particular-use information (general order information), it is possible for the patient's family member to, only by photographing a target patient using the second terminal 20 which has received the particular-use information (general order information), easily and certainly acquire a patient image according to the doctor's instruction (the general order information) without high-level photographing skills, predetermined photographing knowledge or the like, and, at the same time, acquire a medical reference image in a form of an image data file to which medical metadata is attached.

The medical image management system 1 of the present embodiment enables not only image information as patient information acquired by a medical worker but also image information acquired by a general person (a non-medical worker) other than a medical worker to be handled as a medical image or a medical reference image as patient information, and makes it possible to enhance convenience of medical actions.

In this case, since it is possible to create an image data file in a data format compliant with DICOM, which is in such a form that medical metadata including medical basic information and the like included in general order information is associated with an image data file, at the time of photographing an image, it is possible to prevent mistaken selection of an image data file and the like, and it is also possible to improve reliability of information. Further, it becomes easy to manage valuable image information.

Since the second information terminal apparatus (the second terminal 20) is adapted to create an image data file in such a form that medical metadata (accompanying information) including medical basic information and the like included in general order information is associated with an acquired image data file, it is possible to, for example, even if the image data file is transferred to another device, easily identify which kind of image data file the image data file is only by referring to the accompanying information. For example, since a unique number (a number referred to as an order number or the like) included in general order information is associated with a patient ID, an examination ID and the like, the unique number facilitates identification of a patient, and association with a series of examination studies and particular disorder/disease examinations becomes possible.

Note that though description has been made with a general portable image pickup apparatus (camera), a smartphone, a tablet PC or the like provided with an image pickup function and a communication function as an example of one form of the information terminal apparatus (10 or 20) in the one embodiment describe above, the form of an information terminal apparatus applicable to the present invention is not limited to the above forms.

For example, in recent years, a camera system (a network camera system called a so-called watching (monitoring) camera, a web camera or the like) has been in practical use and widespread in which it is enabled to always watch over (monitor) a situation of a target patient or the like to be photographed from a remote location, by installing a camera at a predetermined place (for example, the target patient's home or a hospital room in a hospital), remotely operating the camera via a network to acquire photographed images at predetermined time intervals, and transferring the acquired photographed images to another apparatus (such as a file server) via the network.

A camera included in a network camera system of this kind can be applied as the information terminal apparatus of the present invention.

Though a system used in the medical field is illustrated in the one embodiment described above, the present invention is not limited to the example but can be easily applied to fields other than the medical field, for example, a video field and a scientific research field.

In other words, it is possible to construct a system capable of associating accompanying information including particular-use information created by a specialist in each of various kinds of fields with an image data file acquired by an ordinary person through a predetermined procedure, similarly to the one embodiment described above.

In that case, by recording an image data file obtained by attaching accompanying information including particular-use information created by a specialist to a predetermined file sever to obtain a database, it is possible to secure reliability of each image data file, and thereby contribute to improvement of reliability of an image database in each field.

Note that, as for each processing sequence described in the one embodiment described above, change in a procedure can be permitted unless contrary to the nature of the processing sequence. Therefore, for each processing sequence described above, it is possible, for example, to change execution order of respective processing steps, cause a plurality of processing steps to be executed at the same time, cause the order of the respective steps to differ each time a series of processing sequences is executed. Even if description is made on an operation flow in Claims, the specification and the drawings using “first”, “next” and the like for convenience, it does not mean that it is indispensable to implement the operation flow in that order. It goes without saying that, at each of steps constituting each operation flow, a part that does not influence the essence of the invention can be appropriately omitted.

Among the techniques described here, many of the controls and functions mainly described using a flowchart can be often set by a software program, and the above controls and functions can be realized by a computer reading and executing the software program. The software program is electronic data, the whole or part of which is recorded in a recording medium, a storage portion or the like, more specifically, for example, a portable medium such as a flexible disk, a CD-ROM and a non-volatile memory or a storage medium such as a hard disk and a volatile memory in advance during a product manufacturing process as a computer program product. Aside from the above, the software program can be distributed or provided at product shipment, or can be distributed or provided via portable media or communication lines. Even after product shipment, a user can cause the program software to operate by downloading the software program via a communication network or the Internet himself/herself and installing the software program into a computer, or by installing the program software into the computer from a storage medium, and, thereby, it is possible to easily realize the medical image management system and the information terminal apparatus of the present embodiment.

Here, description has been made with emphasis on a case where a photographing requester is different from a photographer, and a photographed image is used as a strictly medical image. However, of course, the present invention is not limited to medical use but may be applicable to any other field where management of such a photographing request and a photographed image is strictly performed. For example, for images to be recorded at a construction site or images to be recorded at a farmland, there are similar needs.

It is also possible to appropriately replace a part configured with a program with a circuit. In the above description of the one embodiment, each part expressed as “a portion” (a section or a unit) may be configured with a dedicated circuit or a combination of a plurality of general-purpose circuits. If necessary, the part may be configured with a processor such as a microcomputer or a CPU that operates according to software programmed in advance, or a combination of sequencers such as FPGAs. Such design is also possible that a part or all of control of the part is undertaken by an external apparatus. In this case, a wired or wireless communication circuit is interposed. Communication can be performed via Bluetooth (registered trademark), WiFi (registered trademark), a telephone line or the like, and can also be performed via USB. Dedicated circuits, general-purpose circuits and controlling portions may be integrally configured as an ASIC. A part that is mechanically position-controlled (for example, a moving portion) is configured with various actuators and, if necessary, a coupling mechanism for movement, and the actuators are operated by drive circuits. The drive circuits are also controlled by a microcomputer or an ASIC according to a particular program. Such control may be corrected or adjusted in detail by information outputted by various kinds of sensors or peripheral circuits of the various kinds of sensors.

The present invention is not limited to the one embodiment described above, and it is, of course, possible to make various modifications and applications within a range not departing from the spirit of the invention. Furthermore, the above one embodiment includes inventions at various stages, and various inventions can be extracted by an appropriate combination among a plurality of constituent features disclosed. For example, even if some constituent features are deleted from all the constituent features shown in the above one embodiment, a configuration obtained after deleting the constituent features can be extracted as an invention if the problem to be solved by the invention can be solved, and the advantageous effects of the invention can be obtained. Furthermore, components of different embodiments may be appropriately combined. The present invention is limited only by accompanying claims and not restricted by a particular practiced aspect of the present invention.

1. A medical image management system comprising:
an information terminal apparatus configured to transmit medical-related particular-use information about imaging of a particular patient as a part of a physician ordered medical study;
an image pickup apparatus configured to acquire image data; and
a processor including hardware configured to generate a medical image data file by associating (1) medical service-related data included in or derived from the medical-related particular-use information with (2) the image data.

2. The medical image management system according to claim 1, wherein the processor derives the medical service related data from the medical-related particular-use information by classifying at least one of various kinds of information about an examination, such as information on “when”, “where”, “who”, “for whom”, “with which kind of apparatus” and “which kind of examination”, from the medical-related particular-use information.

3. The medical image management system according to claim 1, wherein the medical image data file generated by the processor includes information about whether photographing by the image pickup apparatus, based on the medical-related particular-use information, has been performed or not.

4. The medical image management system according to claim 1, wherein the processor includes a function of judging whether photographing by the image pickup apparatus, based on the medical-related particular-use information, is possible or not.

5. The medical image management system according to claim 1, wherein the processor is provided in the image pickup apparatus.

6. The medical image management system according to claim 1, wherein the processor is provided in a file server connected via a network, between the information terminal apparatus and the image pickup apparatus.

7. The medical image management system according to claim 1, wherein the medical image data file includes an identification of a referring physician.

8. The medical image management system according to claim 7, wherein the identification of the referring physician was included in a general order associated with the medical-related particular-use information.

9. The medical image management system according to claim 1, wherein the medical image data file includes an identification of a person or organization operating the image pickup apparatus to acquire the image data.

10. The medical image management system according to claim 9, wherein the identification of a person or organization operating the image pickup apparatus to acquire the image data was included in a general order associated with the medical-related particular-use information.

11. The medical image management system according to claim 1, wherein the medical image data file includes an identification of a patient to be imaged by the image pickup apparatus to acquire the image data.

12. The medical image management system according to claim 11, wherein the identification of a patient was included in a general order associated with the medical-related particular-use information.

13. The medical image management system according to claim 1, wherein the medical image data file includes (1) an identification of a physician requested study involving the acquisition of the image data by the image pickup apparatus, and (2) required attributes of the image pickup apparatus for performing the physician requested study.

14. The medical image management system according to claim 13, wherein
(1) the identification of a physician requested study involving the acquisition of the image data by the image pickup apparatus, and (2) the required attributes of the image pickup apparatus for performing the physician requested study were included in a general order associated with the medical-related particular-use information.

15. The medical image management system according to claim 1 wherein the medical-related particular-use information includes settings to be applied to the image pickup apparatus.

16. The medical image management system according to claim 1 wherein the medical-related particular-use information includes instructions specifying how to operate the image pickup apparatus during acquisition of the image data by the image pickup apparatus.

17. A medical image acquisition apparatus comprising:
a communication circuit configured to receive medical-related particular-use information; and
a processor including hardware, wherein the processor generates a medical image data file by associating (1) image data acquired by an image pickup device according to the medical-related particular-use information with (2) medical service-related data included in or derived from the medical-related particular-use information.

18. The medical image acquisition apparatus according to claim 17, further comprising:
the image pickup device.

19. A medical image data file acquisition method comprising:
transmitting medical-related particular-use information about a particular individual;
receiving the medical-related particular-use information;
performing an image pickup operation according to the medical-related particular-use information to acquire a image data in which image data and general metadata are associated with each other;
generating medical metadata based on the medical-related particular-use information; and
generating a medical image data file by associating (1) the medical metadata with (2) the image data.

20. A medical image management server configured to perform a communication control method comprising:
receiving, from an information terminal apparatus configured to transmit medical-related particular-use information about a particular individual, the medical-related particular-use information;
responsive to receiving the medical-related particular-use information, transmitting to an image pickup apparatus, an instruction to have the image pickup apparatus perform an image pickup operation according to the received medical-related particular-use information; and
receiving from the image pickup apparatus, image data obtained by the image pickup operation performed by the image pickup apparatus.
